(19) United States
(12) Patent Application Publication
(10) Pub. No.: US 2023/0405018 A1
(43) Pub. Date: Dec. 21, 2023

(54) FLUMAZENIL FORMULATIONS FOR SUBCUTANEOUS INJECTION AND METHODS OF TREATMENT USING GABA RECEPTOR MODULATORS

(71) Applicant: Bexson Biomedical, Inc., Santa Barbara, CA (US)

(72) Inventors: Jeffrey BECKER, Santa Barbara, CA (US); Gregg PETERSON, Santa Barbara, CA (US)

(21) Appl. No.: 18/037,515

(22) PCT Filed: Nov. 17, 2021
(86) PCT No.: PCT/US2021/059763

§ 371 Date: May 17, 2023

Related U.S. Application Data
(60) Provisional application No. 63/115,485, filed on Nov. 18, 2020.

(51) Int. Cl. A61K 31/5517; A61K 47/40; A61K 45/06; A61K 9/00

(52) U.S. Cl. CPC A61K 31/5517; A61K 47/40; A61K 45/06; A61K 9/0019

(57) ABSTRACT

Provided herein are subcutaneous formulations of flumazenil that are useful for treating a variety of disease and disorders. The subcutaneous flumazenil formulations provided herein reduce injection site irritation and pain and allow for home administration to enhance patient compliance.

### CROSS REFERENCE

This application claims the benefit of U.S. Provisional Application No. 63/115,485, filed on Nov. 18, 2020. Said application is incorporated herein by reference in its entirety.

### BACKGROUND

Benzodiazepines represent one of most prescribed medication groups, particularly in primary care settings. This frequently results in benzodiazepine dependence in the subjects, treatment of which is often protracted tapering of the benzodiazepine over several weeks or months. This is often associated with significant withdrawal symptoms which can result in patient drop out and return to use. There exists a need for enhanced treatments and therapy regimens to deal with this problem.

### BRIEF SUMMARY OF THE INVENTION

Provided herein are formulations for subcutaneous delivery of flumazenil. Flumazenil is a selective gamma-aminobutyric acid (GABA) receptor antagonist that is specific for that GABA_{A} receptor. Flumazenil's action on this receptor makes it a potentially powerful tool for dealing with symptoms associated with acute and post-acute benzodiazepine withdrawal. However, flumazenil is readily metabolized by the liver, with less than 25% systemic availability after first pass hepatic metabolism. Subcutaneous delivery of flumazenil would thus be desirable, as it could be slowly administered to a patient over time to maintain a near constant concentration of flumazenil in the relevant tissues. However, flumazenil's poor solubility (˜0.1 mg/mL in USP flumazenil preparations) renders it difficult to formulate at a concentration that would be compatible with such use, which would require a high concentration of the drug in a small volume because validated therapies require daily administration of the drug at an amount of at least 2 mg/day. This would thus require a reservoir of approximately 20 mL for a single day of treatment, which is not compatible with realistic medical product expectations surrounding cost considerations, medical waste, patient usability and patient comfort in a 30 day wearable infusion device protocol. The formulations of flumazenil provided herein have elevated concentrations of flumazenil, thus enabling continuous or near continuous administration of flumazenil over a long period of time from a single, small volume reservoir containing device. Such an approach can also be applied to other GABA receptor modulators for treatment of a variety of other conditions, including alcohol dependence, sedative dependence, panic disorders, generalized anxiety disorders, post-traumatic stress disorders, mood disorders, major depression, dysthymia, chronic suicidality, anxiety disorder NOS, obsessive compulsive disorder, eating disorder NOS, anorexia nervosa, bulimia nervosa, intermittent explosive disorder, a sleep disorder, insomnia NOS, a pain disorder, a chronic pain disorder, idiopathic hypersomnia, and narcolepsy.

In one aspect, provided herein, is a pharmaceutical composition, comprising: (i) flumazenil, or an isotopic variant thereof or a pharmaceutically acceptable salt, solvate or hydrate thereof and (ii) at least one pharmaceutically acceptable excipient, wherein the pharmaceutical composition is in a form for dosing or administration by subcutaneous injection; and wherein the concentration of flumazenil in the pharmaceutical composition is greater than 0.7 mg/mL. In some embodiments, the at least one pharmaceutically acceptable excipient comprises a complexing agent. In some embodiments, the complexing agent is a substituted or unsubstituted cyclodextrin. In some embodiments, the complexing agent is a cyclodextrin substituted with at least one acidic functional group. In some embodiments, the complexing agent is a sulfobutyl-ether-beta-cyclodextrin (SBEBCD). In some embodiments, the complexing agent acts as a counterion to at least a portion of the flumazenil, wherein the portion of the flumazenil is protonated. In some embodiments, the complexing agent is a cyclodextrin substituted with at least one polar functional group. In some embodiments, the complexing agent is a hydroxypropyl-beta-cyclodextrin (HPBCD). In some embodiments, the molar ratio of complexing agent to flumazenil is from about 1:10 to about 10:1. In some embodiments, the pharmaceutical composition further comprises a base, a buffer, or a combination thereof. In some embodiments, the pharmaceutical composition further comprises an emulsifying agent, a surfactant, a solubilizing agent, a co-solvent or a combination thereof. In some embodiments, the co-solvent is ethanol, propylene glycol, tween 20, tween 80, or glycerin. In some embodiments, the pharmaceutical composition has a pH>about 4. In some embodiments, the pharmaceutical composition has a pH of about 4 to about 7. In some embodiments, the pharmaceutical composition has a pH of about 5 to about 8. In some embodiments, the concentration of flumazenil in the pharmaceutical composition is at least about 1 mg/mL. In some embodiments, the concentration of flumazenil in the pharmaceutical composition is at least about 5 mg/mL. In some embodiments, the concentration of flumazenil in the pharmaceutical composition is at most about 10 mg/mL. In some embodiments, the pharmaceutical composition further comprises a preservative. In some embodiments, the pharmaceutical composition has an osmolality of from about 300 mOsm/kg to about 500 mOsm/kg. In some embodiments, the pharmaceutical composition has an osmolality of less than about 500 mOsm/kg. In some embodiments, the pharmaceutical composition is adapted to be delivered to a subject over a period of at least two days. In some embodiments, the pharmaceutical composition is adapted to deliver a daily dose of at least 1 mg of flumazenil. In some embodiments, the pharmaceutical composition is adapted to be delivered by a wearable device. In some embodiments, the wearable device has a reservoir of the pharmaceutical composition of less than 10 mL.

In another aspect, provided herein, is a method of treating benzodiazepine dependence, withdrawal, or toxicity in a subject in need thereof, the method comprising administering a therapeutically effective amount of a pharmaceutical composition provided herein. In some embodiments, the benzodiazepine withdrawal is acute withdrawal or post-acute withdrawal.

In another aspect is a method of treating alcohol dependence, withdrawal, or toxicity, sedative dependence, withdrawal, or toxicity, hypnotic, withdrawal, dependence or toxicity, anxiolytic dependence, withdrawal, or toxicity, panic disorder, generalized anxiety disorder, post-traumatic stress disorder (PTSD), idiopathic hypersomnia, narcolepsy, a mood disorders, major depression, dysthymia, chronic suicidality, anxiety disorder NOS, obsessive compulsive disorder, eating disorder NOS, anorexia nervosa, bulimia nervosa, intermittent explosive disorder, a sleep disorder, insomnia NOS, a pain disorder, or a chronic pain disorder in a subject in need thereof, the method comprising administering a therapeutically effective amount of a pharmaceutical composition provided herein.

In one aspect is a method of treating a disease or disorder in a subject in need thereof, comprising administering to the subject a therapeutically effective amount of flumazenil, wherein the flumazenil is administered by subcutaneous injection in an amount of at least 0.5 mg/day from a single device for a period of time of at least 2 days, wherein the single device has a reservoir volume less than 30 mL. In some embodiments, the disease or disorder is benzodiazepine dependence, withdrawal, or toxicity, alcohol dependence, withdrawal, or toxicity, sedative dependence, withdrawal, or toxicity, hypnotic dependence, withdrawal, or toxicity, anxiolytic dependence, withdrawal, or toxicity, panic disorder, generalized anxiety disorder, post-traumatic stress disorder (PTSD), idiopathic hypersomnia, narcolepsy, a mood disorder, major depression, dysthymia, chronic suicidality, anxiety disorder NOS, obsessive compulsive disorder, eating disorder NOS, anorexia nervosa, bulimia nervosa, intermittent explosive disorder, a sleep disorder, insomnia NOS, a pain disorders, or a chronic pain disorder. In some embodiments, the disease or disorder is benzodiazepine dependence, withdrawal, or toxicity. In some embodiments, the benzodiazepine withdrawal is acute withdrawal or post-acute withdrawal. In some embodiments, the flumazenil is administered as a pharmaceutical composition having a concentration of flumazenil of greater than 0.7 mg/mL. In some embodiments, the flumazenil is administered in an amount of at least 0.5 mg/day, at least 1.0 mg/day, at least 1.5 mg/day, at least 2 mg/day, at least 3 mg/day, at least 4 mg/day, at least 5 mg/day, at least 7.5 mg/day, or at least 10 mg/day. In some embodiments, the method further comprises administering to the subject a therapeutically effective amount of flumazenil from one or more additional single devices, wherein each additional single device is administered to the subject after another iteration of the period of time. In some embodiments, the total time of administration is at least 10 days, at least 15 days, at least 20 days, at least 25 days, or at least 30 days. In some embodiments, the reservoir volume is less than 10 mL. In some embodiments, the reservoir volume is less than 5 mL. In some embodiments, the flumazenil is administered continuously. In some embodiments, the flumazenil is administered at a rate of at least about 40 μg/hr, at least about 60 μg/hr, at least about 80 μg/hr, at least about 100 μg/hr, at least about 150 μg/hr, at least about 200 μg/hr, or at least about 250 μg/hr.

In another aspect, provided herein, is a pharmaceutical composition, comprising: (i) a pharmaceutical compound, wherein the pharmaceutical compound is a GABA_{A} receptor antagonist or modulator, a GABA receptor agonist, a GABA receptor partial agonist, a GABA_{A} receptor negative allosteric modulator or inverse agonist, or salvinorin A, or an isotopic variant thereof; or a pharmaceutically acceptable salt, solvate or hydrate thereof; and (ii) at least one pharmaceutically acceptable excipient, wherein the at least one pharmaceutically acceptable excipient comprises a cyclodextrin substituted with at least one acidic functional group or a conjugate base thereof or a cyclodextrin substituted with at least one polar functional group, wherein the pharmaceutical composition is in a form for dosing or administration by subcutaneous injection. In some embodiments, the pharmaceutical compound is a GABA_{A} receptor antagonist or modulator. In some embodiments, the pharmaceutical compound is flumazenil or pentylenetetrazol. In some embodiments, the pharmaceutical compound is a GABA receptor agonist. In some embodiments, the pharmaceutical compound is muscimol, thiomuscimol, or gaboxadol. In some embodiments, the pharmaceutical compound is a GABA receptor partial agonist. In some embodiments, the pharmaceutical compound is bretazenil, imidazenil, FG 8205 (7-chloro-5-methyl-3-(5-propan-2-yl-1,2,4-oxadiazol-3-yl)-4H-imidazo[1,5-a][1,4]benzodiazepin-6-one), abecarnil, NS 2710 (1-[1-[3-(3-pyridyl)phenyl]benzimidazol-5-yl]ethanone O-ethyloxime), RWJ-51204 (5-ethoxymethyl-7-fluoro-3-oxo-1,2,3,5-tetrahydrobenzo[4,5] imidazo[1,2a]pyridine-4-N-(2-fluorophenyl)carboxamide), or premazepam. In some embodiments, the pharmaceutical compound is a GABA_{A} receptor negative allosteric modulator or inverse agonist. In some embodiments, the pharmaceutical compound is bemegride, flurothyl, or pentylenetetrazol. In some embodiments, the pharmaceutical compound is an a5 subunit containing GABA_{A} receptor selective compound. In some embodiments, the pharmaceutical compound is basmisanil, α5IA (3-(5-methylisoxazol-3-yl)-6-[(1-methyl-1H-1,2,3-triazol-4-yl)methoxy [1,2,4]triazolo[3,4-a]phthalazine), L-655,708, MRK-016, PWZ-029, a Pyridazine, Ro4938581, TB-21007, FG-7142 (N-Methyl-9H-pyrido[5,4-b]indole-3-carboxamide), Ro16-0154 (ethyl 7-iodanyl-5-methyl-6-oxo-4H-imidazo[1,5-a][1,4]benzodiazepine-3-carboxylate), Radequinil, Ro15-4513 (Ethyl-8-azido-5,6-dihydro-5-methyl-6-oxo-4H-imidazo-1,4-benzodiazepine-3-carboxylate), Sarmazenil, Suritozole, Terbequinil, or ZK-93426 (ethyl-5-isopropoxy-4-methyl-beta-carboline-3-carboxylate). In some embodiments, the pharmaceutical compound is salvinorin A.

In an aspect, provided herein, is a method of treating a disease or disorder in a subject in need thereof, comprising administering to a subject in need thereof a therapeutically effective amount of pharmaceutical compounds, wherein the pharmaceutical compound is a GABA_{A} receptor antagonist or modulator, a GABA receptor agonist, a GABA receptor partial agonist, a GABA_{A} receptor negative allosteric modulator or inverse agonist, or salvinorin A, wherein the pharmaceutical compound is administered by subcutaneous injection from a single device for a period of time of at least 2 days, wherein the single device has a reservoir volume less than 30 mL. In some embodiments, the disease or disorder is benzodiazepine dependence, withdrawal, or toxicity, alcohol dependence, withdrawal, or toxicity, sedative dependence, withdrawal, or toxicity, hypnotic dependence, withdrawal, or toxicity, anxiolytic dependence, withdrawal, or toxicity, panic disorder, generalized anxiety disorder, post-traumatic stress disorder (PTSD), idiopathic hypersomnia, narcolepsy, a mood disorder, major depression, dysthymia, chronic suicidality, anxiety disorder NOS, obsessive compulsive disorder, eating disorder NOS, anorexia nervosa, bulimia nervosa, intermittent explosive disorder, a sleep disorder, insomnia NOS, a pain disorder, or a chronic pain disorder. In some embodiments, the benzodiazepine withdrawal is acute withdrawal or post-acute withdrawal.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURE(S)

FIG. 1 shows a phase solubility curve with captisol and flumazenil.

### DETAILED DESCRIPTION

Provided herein are, for example, compositions comprising flumazenil with reduced irritant effect to subcutaneous tissues and/or dermal tissues. In certain aspects, the compositions comprising flumazenil are formulated for subcutaneous administration. In some aspects, the compositions are formulated to be administered to a subject over a period of at least two days, preferably from a wearable device. Also provided herein are, for example, methods of treating or preventing benzodiazepine dependence, withdrawal, and/or toxicity, and other disease and disorders with flumazenil and other GABA receptor modulators.

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O—is equivalent to —OCH₂—.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

All average molecular weights of polymers are weight-average molecular weights, unless otherwise specified.

As used herein, “individual” (as in the subject of the treatment) means both mammals and non-mammals. Mammals include, for example, humans; non-human primates, e.g. apes and monkeys; and non-primates, e.g. dogs, cats, cattle, horses, sheep, and goats. Non-mammals include, for example, fish and birds.

The terms “disease,” “disorder,” or “condition” refer to a state of being or health status of a patient or subject capable of being treated with the compounds or methods provided herein. The disease may be a disease of dependence or withdrawal, such as benzodiazepine dependence or withdrawal, as well as toxicity. The disease may be a mood disorder. In some further instances, “mental or psychiatric disorder” refers to human mental or psychiatric disorders including major depressive disorder, treatment resistant major depressive disorder, dysthymia, Suicidality, Suicidal Ideation, bipolar I disorder, bipolar II disorder, post-traumatic stress disorder (PTSD), a substance-related disorder (e.g., Cannabis dependence or withdrawal, barbiturate dependence or withdrawal, benzodiazepine dependence or withdrawal, amphetamine dependence or withdrawal, opioid dependence or withdrawal, alcohol dependence or withdrawal, or cocaine dependence or withdrawal.

The expression “withdrawal” when used to describe a disease refers to both the well characterized early signs and symptoms in the initial phase of discontinuation or taper of a substance an individual is physically or psychologically dependent upon, as well as the later, long term or lingering signs and symptoms after the immediate period of discontinuation or during a long term slow discontinuation or taper which are sometimes called “post-acute” withdrawal.”

The expression “effective amount,” when used to describe therapy to an individual suffering from a disorder, refers to the amount of a compound (e.g. flumazenil) described herein that is effective to act on or modulate (e.g. inhibit) GABA receptors, such as the GABA_{A} receptor, in the individual's tissues wherein, wherein such modulating or other action occurs to an extent sufficient to produce a beneficial therapeutic effect.

“Substantially” as the term is used herein means completely or almost completely. For example, a composition that is “substantially free” of a component either has none of the component or contains such a trace amount that any relevant functional property of the composition is unaffected by the presence of the trace amount. For example, a compound that is “substantially pure” has only negligible traces of impurities present.

All chiral, diastereomeric, and/or racemic forms of a structure are intended, unless a particular stereochemistry or isomeric form is specifically indicated. Compounds described herein can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the present disclosure.

The inclusion of an isotopic form of one or more atoms in a molecule that is different from the naturally occurring isotopic distribution of the atom in nature is referred to as an “isotopically labeled form” of the molecule. All isotopic forms of atoms are included as options in the composition of any molecule, unless a specific isotopic form of an atom is indicated. For example, any hydrogen atom or set thereof in a molecule can be any of the isotopic forms of hydrogen, e.g., protium (¹H), deuterium (²H), or tritium (³H) in any combination. Similarly, any carbon atom or set thereof in a molecule can be any of the isotopic form of carbons, such as ¹¹C, ¹²C, ¹³C, or ¹⁴C, or any nitrogen atom or set thereof in a molecule can be any of the isotopic forms of nitrogen, such as ¹³N, ¹⁴N, or ¹⁵N. A molecule can include any combination of isotopic forms in the component atoms making up the molecule, the isotopic form of every atom forming the molecule being independently selected. In a multi-molecular sample of a compound, not every individual molecule necessarily has the same isotopic composition. For example, a sample of a compound can include molecules containing various different isotopic compositions, such as in a tritium or ¹⁴C radiolabeled sample where only some fraction of the set of molecules making up the macroscopic sample contains a radioactive atom. It is also understood that many elements that are not artificially isotopically enriched themselves are mixtures of naturally occurring isotopic forms, such as ¹⁴N and ¹⁵N, ³²S and ³⁴S, and so forth. A molecule as recited herein is defined as including isotopic forms of all its constituent elements at each position in the molecule. As is well known in the art, isotopically labeled compounds can be prepared by the usual methods of chemical synthesis, except substituting an isotopically labeled precursor molecule. The isotopes, radiolabeled or stable, can be obtained by any method known in the art, such as generation by neutron absorption of a precursor nuclide in a nuclear reactor, by cyclotron reactions, or by isotopic separation such as by mass spectrometry. The isotopic forms are incorporated into precursors as required for use in any particular synthetic route. For example, ¹⁴C and ³H can be prepared using neutrons generated in a nuclear reactor. Following nuclear transformation, ¹⁴C and ³H are incorporated into precursor molecules, followed by further elaboration as needed.

A “hydrate” is a compound that exists in a composition with water molecules. The composition can include water in stoichiometric quantities, such as a monohydrate or a dihydrate, or can include water in random amounts. As the term is used herein a “hydrate” refers to a solid form, e.g., a compound in water solution, while it may be hydrated, is not a hydrate as the term is used herein.

A “solvate” is a similar composition except that a solvent other that water replaces the water. For example, methanol or ethanol can form an “alcoholate”, which can again be stoichiometic or non-stoichiometric. As the term is used herein a “solvate” refers to a solid form, e.g., a compound in solution in a solvent, while it may be solvated, is not a solvate as the term is used herein.

A “prodrug” as is well known in the art is a substance that can be administered to a patient where the substance is converted in vivo by the action of biochemicals within the patient's body, such as enzymes, to the active pharmaceutical ingredient. Examples of prodrugs include esters of carboxylic acid groups, which can be hydrolyzed by endogenous esterases as are found in the bloodstream of humans and other mammals. Further examples of prodrugs include boronate esters which can be hydrolyzed under physiological conditions to afford the corresponding boronic acid. Conventional procedures for the selection and preparation of suitable prodrug derivatives are described, for example, in “Design of Prodrugs”, ed. H. Bundgaard, Elsevier, 1985.

In various embodiments, a compound as shown in any of the Examples, or among the exemplary compounds, is provided.

Provisos may apply to any of the disclosed categories or embodiments wherein any one or more of the other above disclosed embodiments or species may be excluded from such categories or embodiments.

### Isomerism in Compounds Described Herein

### Optical Isomerism

It will be understood that when compounds of the present disclosure contain one or more chiral centers, the compounds may exist in, and may be isolated as pure enantiomeric or diastereomeric forms or as racemic mixtures. The present disclosure therefore includes any possible enantiomers, diastereomers, racemates or mixtures thereof of the compounds described herein.

The isomers resulting from the presence of a chiral center comprise a pair of non-superimposable isomers that are called “enantiomers.” Single enantiomers of a pure compound are optically active, e.g., they are capable of rotating the plane of plane polarized light. Single enantiomers are designated according to the Cahn-Ingold-Prelog system. The priority of substituents is ranked based on atomic weights, a higher atomic weight, as determined by the systematic procedure, having a higher priority ranking. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example below, the Cahn-Ingold-Prelog ranking is A>B>C>D. The lowest ranking atom, D is oriented away from the viewer.

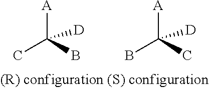

The present disclosure is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

“Isolated optical isomer” means a compound which has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well-known chiral separation techniques. According to one such method, a racemic mixture of a compound described herein, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; e.g., the R and S configurations for each asymmetric center. Therefore, single stereochemical isomers as well as enantiomeric and diastereomeric mixtures of the present compounds are within the scope of the disclosure.

“Alkyl” refers to a straight or branched hydrocarbon chain radical consisting solely of carbon and hydrogen atoms, which may optionally be unsaturated with one or more double or triple bonds, and preferably having from one to fifteen carbon atoms (i.e., C₁-C₁₅ alkyl). In certain embodiments, an alkyl comprises one to six carbon atoms (i.e., C₁-C₆ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless otherwise specified, the term “alkyl” and its equivalents encompass linear, branched, and/or cyclic alkyl groups. In some instances, an “alkyl” comprises both cyclic and acyclic (linear and/or branched) alkyl components.

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents may be one or more and the same or different for appropriate organic compounds.

Substituents may include any substituent, for example, a halogen, a hydroxyl, a carbonyl (such as an oxo (═O), a carboxyl, an alkoxycarbonyl, a formyl, or an acyl), a thiocarbonyl (such as a thioxo (═S), a thioester, a thioacetate, or a thioformate), an alkoxyl, a phosphoryl, a phosphate, a phosphonate, a phosphinate, an amino, an amido, an amidine, an imine, an oximo, a hydrazino, a cyano, a nitro, an azido, a sulfhydryl, an alkyl, an alkylthio, a sulfate, a sulfonate, a sulfamoyl, a sulfonamido, a sulfonyl, an aralkyl, a carbocycle, a heterocycle, a cycloalkyl, a heterocycloalkyl, an aromatic and heteroaromatic moiety.

As used herein, an “acidic functional group” or similar term (e.g. “acidic functionality”) refers to a chemical moiety which contains at least one dissociable proton (or isotopic variant thereof), as well as to the conjugate base of such a group unless otherwise specified. In certain embodiments, the dissociable proton dissociates from the chemical moiety at a pH common in aqueous systems (e.g. pHs from about 1 to about 14). In certain preferred embodiments, the dissociable proton dissociates from the chemical moiety in an aqueous system at a pH of less than 7 (e.g. having a pKa value of less than 7, such as a pKa of less than 6, less than 5, less than 4, less than 3, less than 2, or less than 1). As is understood by those in the art, whether an acidic functional group contains the dissociable proton will depend on the conditions of the system in which the chemical moiety is present (e.g., the pH of an aqueous system containing molecule with the acidic functional group or the presence of any base molecule). As such, the term “acidic functional group” (or reference to a specific acidic functional group such as a carboxylic acid or a sulfonic acid) as used herein is intended to cover the protonated version of the moiety, the deprotonated version of the moiety, and any salt of the moiety, unless otherwise specified.

Unless otherwise stated, structures depicted herein are also meant to include compounds which differ only in the presence of one or more isotopically enriched atoms (e.g., isotopic variant(s)). For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

A “salt,” as is well known in the art, includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH 4⁺ or the cations of various amines, including tetraalkyl ammonium salts such as tetramethylammonium, or other cations such as trimethylsulfonium, and the like. The terms “pharmaceutically acceptable salts” and/or or “pharmacologically acceptable salts” are meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents. In certain embodiments, compounds of the present disclosure contain both basic and acidic functionalities that allow the compounds to be converted into either base or acid addition salts. The neutral forms of the compounds may be regenerated by contacting the salt with a base or acid and isolating the parent compound in a conventional manner. The parent form of the compounds differs from the various salt forms in certain physical properties, such as solubility in polar solvents, but, unless specifically indicated, the salts disclosed herein are equivalent to the parent form of the compound for the purposes of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of a compound to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, complexing agents (e.g. cyclodextrins), binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The terms “treating” or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In certain embodiments, treating is preventing. In certain embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (e.g., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of a compound described herein. The administering step may consist of a single administration or may include a series of administrations. The length of the treatment period depends on a variety of factors, such as the severity of the condition, the age of the patient, the concentration of the compound, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

The term “prevent” refers to a decrease in the occurrence of disease symptoms in a patient. As indicated above, the prevention may be complete (no detectable symptoms) or partial, such that fewer symptoms are observed than would likely occur absent treatment. In certain embodiments, prevent refers to slowing the progression of the disease, disorder or condition or inhibiting progression thereof to a harmful or otherwise undesired state.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington. The Science and Practice of Pharmacy, 20th Edition, 2003, Gennaro, Ed., Lippincott, Williams & Wilkins). The therapeutically effective amount can be ascertained by measuring relevant physiological effects, and it can be adjusted in connection with the dosing regimen and diagnostic analysis of the subject's condition, and the like. By way of example, measurement of the serum level of an inhibitor (or, e.g., a metabolite thereof) at a particular time post-administration may be indicative of whether a therapeutically effective amount has been administered.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan. Adjusting the dose to achieve maximal therapeutic window efficacy or toxicity in humans based on the methods described above and other methods is well within the capabilities of the ordinarily skilled artisan.

The term “therapeutically effective amount,” as used herein, refers to that amount of the therapeutic agent sufficient to ameliorate the disorder, as described herein. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means subcutaneous (i.e., “SC,” “subQ,” or “SQ”) administration, oral administration, administration as a suppository, topical contact or administration, intravenous, parenteral, intraperitoneal, intramuscular, intraosseous, intralesional, intrathecal, intracranial, intranasal, epidural, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-cancer agent, chemotherapeutic, or treatment for a neurodegenerative disease). The compound of the disclosure can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compositions of the present disclosure can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present disclosure can also be delivered as microspheres for slow release in the body. For example, microspheres can be administered via intradermal injection of drug-containing microspheres, which slowly release subcutaneously (see Rao, J. Biomater Sci. Polym. Ed. 7.623-645, 1995; as biodegradable and injectable gel formulations (see, e.g., Gao Pharm. Res. 12.857-863, 1995); or, as microspheres for oral administration (see, e.g., Eyles, J. Pharm. Pharmacol. 49.669-674, 1997). In another embodiment, the formulations of the compositions of the present disclosure can be delivered by the use of liposomes which fuse with the cellular membrane or are endocytosed, e.g., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13.293-306, 1996; Chonn, Curr. Opin. Biotechnol. 6.698-708, 1995; Ostro, Am. J. Hosp. Pharm. 46.1576-1587, 1989). The compositions of the present disclosure can also be delivered as nanoparticles.

By “co-administer” it is meant that a composition described herein is administered at the same time, just prior to, or just after the administration of one or more additional therapies. The compounds of the disclosure can be administered alone or can be coadministered to the patient. Coadministration is meant to include simultaneous or sequential administration of the compounds individually or in combination (more than one compound). The compositions of the present disclosure can be delivered transdermally, by a topical route, or formulated as applicator sticks, solutions, suspensions, emulsions, gels, creams, ointments, pastes, jellies, paints, powders, and aerosols.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating a mental or psychiatric disorder, a mood disorder, a neurological condition or disorder, a metabolic disorder (e.g., type 2 diabetes mellitus and/or complications thereof), endometriosis, glaucoma, pain, or an inflammatory disorder.

In some embodiments, co-administration includes administering one active agent within 0.5, 1, 2, 4, 6, 8, 10, 12, 16, 20, 24 hours, 2 days, 4 days, 1 week or 1 month of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, e.g., preparing a single pharmaceutical composition including both active agents. In other embodiments, the active agents can be formulated separately. In another embodiment, the active and/or adjunctive agents may be linked or conjugated to one another. In some embodiments, the compounds described herein may be combined with treatments for infections (e.g. bacterial infections), inflammation, and/or vasodilation.

The compounds described herein can be administered to treat a metabolic disease or disorder (e.g., type 2 diabetes mellitus and/or complications thereof), a mental or psychiatric disorder, a mood disorder, a neurological condition or disorder, endometriosis, glaucoma, pain, or an inflammatory disorder. In this regard, the compounds disclosed herein may be administered either alone to treat such diseases or disorders or may be co-administered with another therapeutic agent to treat such diseases or disorders.

The compounds disclosed herein may be co-administered with other active agents including but not limited to antidepressants, antipsychotics, anti-inflammatories, anxiolytics, and/or analgesics.

The compounds (e.g., flumazenil, etc.) disclosed herein may be administered once daily until study reached endpoint, or may be administered by a continuous dose or otherwise administered over a period of time (e.g. by a wearable device according to a pre-programmed protocol designed to maintain a certain concentration in relevant tissues in the subject).

The term “bioavailability (F),” as used herein, refers to the fraction of a dose of drug (e.g., epinephrine) that is absorbed from its site of administration and reaches, in an unchanged form, the systemic circulation. The term “absolute bioavailability” is used when the fraction of absorbed drug is related to its I.V. bioavailability. It may be calculated using the following formula.

$$F = \frac{{AUC}_{extravascular}}{{AUC}_{intravenous}} \times \frac{{Dose}_{intravenous}}{{Dose}_{e x t r a v a s c u l a r}}$$

The term relative bioavailability (Fᵣₑₗ) is used to compare two different extravascular routes of drug administration and it may be calculated using the following formula.

$${F}_{r e l} = \frac{{AUC}_{extravascular 1}}{{AUC}_{extravascular 2}} \times \frac{{Dose}_{extravascular 2}}{{Dose}_{extravascular 1}}$$

The term “clearance (CL),” as used herein, refers to the rate at which a drug is eliminated divided by its plasma concentration, giving a volume of plasma from which drug is completely removed per unit of time. CL is equal to the elimination rate constant (λ) multiplied by the volume of distribution (V d), wherein “V_{d}” is the fluid volume that would be required to contain the amount of drug present in the body at the same concentration as in the plasma. The term “apparent clearance (CL/F),” as used herein, refers to clearance that does not take into account the bioavailability of the drug. It is the ratio of the dose over the AUC.

The term “flumazenil,” as used herein, refers to a compound of the following structure.

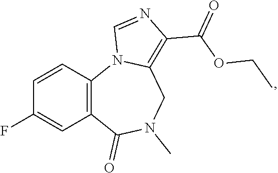 [SMILES: CCOC(=O)c1ncn2c1CN(C)C(=O)c1cc(F)ccc1-2]

or a pharmaceutically acceptable salt, hydrate, or solvate thereof. The CAS registry number for flumazenil is 78755-81-4. Other names for flumazenil include, but are not limited to ethyl 8-fluoro-5,6-dihydro-5-methyl-6-oxo-4H-imidazo[1,5-a][1,4] benzodiazepine-3-carboxylate.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., Spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

Generally, dosage levels of flumazenil in the compositions can range from about 5 μg/kg to about 10 mg/kg, from about 0.5 mg/kg to about 5 mg/kg, from about 1 mg/kg to about 3 mg/kg, or a fixed dose from about 10-100 mg, or 20-75 mg, or 3-60 mg, or 10-250 mg, or 10-400 mg, or an amount greater than 400 mg. In preferred embodiments, the dosages are low, in the range of 0.5-6 mg/day in a subject.

“Substantially pure” indicates that a component makes up greater than about 50% of the total content of the composition, and typically greater than about 60% of the total content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition (percentage in a weight per weight basis).

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway (e.g., MAP kinase pathway).

As defined herein, the terms “activation,” “activate,” “activating,” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc., refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist. In embodiments, an agonist is a molecule that interacts with a target to cause or promote an increase in the activation of the target. In embodiments, activators are molecules that increase, activate, facilitate, enhance activation, sensitize, or up-regulate, e.g., a gene, protein, ligand, receptor, or cell.

The “activity” of a molecule may describe or refer to the binding of the molecule to a ligand or to a receptor; to catalytic activity; to the ability to stimulate gene expression or cell signaling, differentiation, or maturation; to antigenic activity; to the modulation of activities of other molecules; and the like.

The term “osmolality” as described herein is defined as the number of osmoles (Osm) of solute per kilogram of solvent (osmol/kg or Osm/kg).

The term “osmolarity” as described herein is defined is defined as the number of osmoles of solute per liter (L) of solution (osmol/L or Osm/L).

Osmolarity may be calculated from osmolality as follows. osmolarity=osmolality×(ρₛₒₗ-cₐ); where ρₛₒₗ is the density of the solution in g/mL and c a is the (anhydrous) solute concentration in g/mL. Unless expressly stated otherwise, osmolarity is calculated using osmolality according to the preceding formula. Alternatively, osmolarity may be calculated experimentally.

### II. Compositions

Provided herein are pharmaceutical formulations of flumazenil suitable for dosing or administration by subcutaneous injection. Subcutaneously deliverable flumazenil has the advantage over other forms of flumazenil (e.g. IV or IM delivery) in that it can be used outside of a hospital or clinical setting, such as at home by the subject. Other formulations of flumazenil are limited by the low solubility of flumazenil in neutral solutions. Indeed, currently available USP formulations are acidic (pH˜4) and of a very low concentration (˜0.1 mg/mL), which render them unsuitable for subcutaneous administration, as the low pH would likely cause injection site pain and discomfort and the low concentration would require a substantial volume to be delivered over the course of treatment. This high volume requirement has limited use of flumazenil to hospital settings where it can be administered by IV. Additionally, flumazenil has extremely limited oral bioavailability and a very short half-life, making oral formulations of flumazenil unrealistic solutions. The formulations of flumazenil provided herein solve this problem by utilize complexing agents to enhance the solubility of flumazenil sufficiently to allow a sufficiently high concentration of flumazenil to be solubilized in a biocompatible medium. This enables the formulations to be administered by a wearable device having a small reservoir volume, thus enabling the patient to maintain a normal lifestyle during treatment and enhancing patient compliance with the treatment regimen, thus giving the treatment the greatest chance of success (as many treatments using flumazenil, such as for benzodiazepine dependence or withdrawal, are expected to produce added benefit with continued administration for multiple weeks).

In an aspect, provided herein, is a pharmaceutical composition, comprising: (i) flumazenil, or an isotopic variant thereof; or a pharmaceutically acceptable salt, solvate or hydrate thereof; and (ii) at least one pharmaceutically acceptable excipient, wherein the pharmaceutical composition is in a form for dosing or administration by subcutaneous injection; and wherein the concentration of flumazenil in the pharmaceutical composition is greater than 0.7 mg/mL.

In another aspect, provided herein, is a pharmaceutical composition, comprising: (i) flumazenil, or an isotopic variant thereof; or a pharmaceutically acceptable salt, solvate or hydrate thereof; and (ii) at least one complexing agent, wherein the pharmaceutical composition is in a form for dosing or administration by subcutaneous injection; and wherein the concentration of flumazenil in the pharmaceutical composition is greater than 0.7 mg/mL.

Also provided herein is a pharmaceutical composition, comprising (i) a pharmaceutical compound, wherein the pharmaceutical compound is a GABA_{A} receptor antagonist or modulator, a GABA receptor agonist, a GABA receptor partial agonist, a GABA_{A} receptor negative allosteric modulator or inverse agonist, or salvinorin A, or an isotopic variant thereof; or a pharmaceutically acceptable salt, solvate or hydrate thereof; and (ii) at least one pharmaceutically acceptable excipient, wherein the at least one pharmaceutically acceptable excipient comprises a cyclodextrin substituted with at least one acidic functional group or a conjugate base thereof or a cyclodextrin substituted with at least one polar functional group, wherein the pharmaceutical composition is in a form for dosing or administration by subcutaneous injection.

In some embodiments, the at least one pharmaceutically acceptable excipient comprises a complexing agent. In some embodiments, the complexing agent is a cyclodextrin. Cyclodextrins are a family of cyclic oligosaccharides comprising a macrocyclic ring of glucose or other sugar subunits joined by α-1,4 glycosidic bonds. Common forms of cyclodextrins include alpha-cyclodextrins which contain 6 glucose subunits, beta-cyclodextrins which contain 7 glucose subunits, and gamma-cyclodextrins which contain 8 glucose subunits. Cyclodextrins may be substituted or unsubstituted (e.g. have no substituents attached to the glucose monomers).

In some embodiments, the complexing agent is a substituted or unsubstituted cyclodextrin. In some embodiments, the substituted cyclodextrin comprises ionic functional groups. In some embodiments, the ionic functional groups are carboxylate groups, phosphate groups, phosphonate groups, sulfate groups, or sulfonate groups. In some embodiments, cyclodextrin is substituted with sulfonate functional groups. In some embodiments, the cyclodextrin is substituted with at least 1, at least 2, at least 3, at least 4, at least 5, at least 6, at least 7, or at least 8 ionic functional groups. In some embodiments, the cyclodextrin is substituted with from about 2 to about 8 ionic functional groups. In some embodiments, the cyclodextrin is substituted with from about 4 to about 8 ionic functional groups.

In some embodiments, the cyclodextrin is substituted with at least one acidic functional group. In some embodiments, the acidic functional group is a carboxylic acid, a phosphonic acid, or a sulfonic acid. In some embodiments, the acidic functional group is a sulfonic acid. In some embodiments, the cyclodextrin is substituted with at least 1, at least 2, at least 3, at least 4, at least 5, at least 6, at least 7, or at least 8 acidic functional groups. In some embodiments, the cyclodextrin is substituted with from about 2 to about 8 acidic functional groups. In some embodiments, the cyclodextrin is substituted with from about 4 to about 8 acidic functional groups.

In some embodiments, the cyclodextrin is a sulfobutyl-ether substituted cyclodextrin. In some embodiments, the cyclodextrin is a sulfobutyl-ether-alpha-cyclodextrin (SBEACD), sulfobutyl-ether-beta-cyclodextrin (SBEBCD), sulfobutyl-ether-gamma-cyclodextrin (SBEGCD). In some embodiments, the cyclodextrin is sulfobutyl-ether-beta-cyclodextrin (SBEBCD).

In some embodiments, the complexing agent is a free acid version of a cyclodextrin substituted with at least one acidic functional group. In such cases, the complexing agent can act as a counterion to flumazenil which has become protonated. In some embodiments, at least a portion of the flumazenil is protonated. In some embodiments, the free acid version of the cyclodextrin is used in an intermediate step in the preparation of the pharmaceutical formulation. Such a free acid version of an acid substituted cyclodextrin can be used to initially solubilize the flumazenil prior to the addition of other excipients, such as additional buffers which can be used to raise the pH of the intermediate protonated flumazenil, which can result in a soluble complex of flumazenil while mitigating the high osmolality which would result from a direct mixing of a salt form of the cyclodextrin substituted with one or more acidic functional groups (e.g. a sodium salt) at the same concentration.

In some embodiments, the complexing agent is a cyclodextrin substituted with at least one polar functional groups. In some embodiments, the polar functional group is non-ionic. In some embodiments, the polar functional group is a hydroxy-alkyl group, an alkoxy alkyl group, a thioether group, an ester group, an amide group, a sulfonamide group, a sulfonate ester group, or any combination thereof. In some embodiments, the polar functional group is a hydroxyalkyl group. In some embodiments, the polar functional group is a C₂-C₆ hydroxyalkyl group. In some embodiments, the polar functional group is a hydroxyethyl group, a hydroxypropyl group, a hydroxybutyl group, a hydroxypentyl group, or a hydroxyhexyl group. In some embodiments, the polar functional group is a hydroxypropyl group.

In some embodiments, the complexing agent is a hydroxybutyl substituted cyclodextrin. In some embodiments, the complexing agent is a hydroxypropyl-alpha-cyclodextrin (HPACD), a hydroxypropyl-beta-cyclodextrin (HPBCD), or a hydroxypropyl-gamma-cyclodextrin (HPGCD). In some embodiments, the complexing agent is HPBCD.

In some embodiments, the molar ratio of complexing agent to flumazenil is about 1:10 to about 10:1. In some embodiments, the molar ratio of flumazenil to complexing agent is about 1:10 to about 1:5, about 1:10 to about 1:1, about 1:10 to about 2:1, about 1:10 to about 3:1, about 1:10 to about 4:1, about 1:10 to about 5:1, about 1:10 to about 10:1, about 1:5 to about 1:1, about 1:5 to about 2:1, about 1:5 to about 3:1, about 1:5 to about 4:1, about 1:5 to about 5:1, about 1:5 to about 10:1, about 1:1 to about 2:1, about 1:1 to about 3:1, about 1:1 to about 4:1, about 1:1 to about 5:1, about 1:1 to about 10:1, about 2:1 to about 3:1, about 2:1 to about 4:1, about 2:1 to about 5:1, about 2:1 to about 10:1, about 3:1 to about 4:1, about 3:1 to about 5:1, about 3:1 to about 10:1, about 4:1 to about 5:1, about 4:1 to about 10:1, or about 5:1 to about 10:1. In some embodiments, the molar ratio of flumazenil to complexing agent is about 1:10, about 1:5, about 1:1, about 2:1, about 3:1, about 4:1, about 5:1, or about 10:1. In some embodiments, the molar ratio of flumazenil to complexing agent is at least about 1:10, about 1:5, about 1:1, about 2:1, about 3:1, about 4:1, or about 5:1. In some embodiments, the molar ratio of flumazenil to complexing agent is at most about 1:5, about 1:1, about 2:1, about 3:1, about 4:1, about 5:1, or about 10:1. In some embodiments, the complexing agent is HPBCD. In some embodiment, the complexing agent is SBEBCD.

In some embodiments, the concentration of the complexing agent is about 10 mg/mL to about 200 mg/mL. In some embodiments, the concentration of the complexing agent is about 10 mg/mL to about 25 mg/mL, about 10 mg/mL to about 50 mg/mL, about 10 mg/mL to about 75 mg/mL, about 10 mg/mL to about 100 mg/mL, about 10 mg/mL to about 125 mg/mL, about 10 mg/mL to about 150 mg/mL, about 10 mg/mL to about 175 mg/mL, about 10 mg/mL to about 200 mg/mL, about 25 mg/mL to about 50 mg/mL, about 25 mg/mL to about 75 mg/mL, about mg/mL to about 100 mg/mL, about 25 mg/mL to about 125 mg/mL, about 25 mg/mL to about 150 mg/mL, about 25 mg/mL to about 175 mg/mL, about 25 mg/mL to about 200 mg/mL, about 50 mg/mL to about 75 mg/mL, about 50 mg/mL to about 100 mg/mL, about 50 mg/mL to about 125 mg/mL, about 50 mg/mL to about 150 mg/mL, about 50 mg/mL to about 175 mg/mL, about 50 mg/mL to about 200 mg/mL, about 75 mg/mL to about 100 mg/mL, about 75 mg/mL to about 125 mg/mL, about 75 mg/mL to about 150 mg/mL, about 75 mg/mL to about 175 mg/mL, about 75 mg/mL to about 200 mg/mL, about 100 mg/mL to about 125 mg/mL, about 100 mg/mL to about 150 mg/mL, about 100 mg/mL to about 175 mg/mL, about 100 mg/mL to about 200 mg/mL, about 125 mg/mL to about 150 mg/mL, about 125 mg/mL to about 175 mg/mL, about 125 mg/mL to about 200 mg/mL, about 150 mg/mL to about 175 mg/mL, about 150 mg/mL to about 200 mg/mL, or about 175 mg/mL to about 200 mg/mL. In some embodiments, the concentration of the complexing agent is about 10 mg/mL, about 25 mg/mL, about 50 mg/mL, about 75 mg/mL, about 100 mg/mL, about 125 mg/mL, about 150 mg/mL, about 175 mg/mL, or about 200 mg/mL. In some embodiments, the concentration of the complexing agent is at least about 10 mg/mL, about 25 mg/mL, about 50 mg/mL, about 75 mg/mL, about 100 mg/mL, about 125 mg/mL, about 150 mg/mL, or about 175 mg/mL. In some embodiments, the concentration of the complexing agent is at most about 25 mg/mL, about 50 mg/mL, about 75 mg/mL, about 100 mg/mL, about 125 mg/mL, about 150 mg/mL, about 175 mg/mL, or about 200 mg/mL. In some embodiments, the complexing agent is HPBCD. In some embodiment, the complexing agent is SBEBCD.

In some embodiments, the concentration of HPBCD is about 10 mg/mL to about 300 mg/mL. In some embodiments, the concentration of HPBCD is about 10 mg/mL to about 50 mg/mL, about 10 mg/mL to about 100 mg/mL, about 10 mg/mL to about 150 mg/mL, about 10 mg/mL to about 200 mg/mL, about 10 mg/mL to about 250 mg/mL, about 10 mg/mL to about 300 mg/mL, about 50 mg/mL to about 100 mg/mL, about 50 mg/mL to about 150 mg/mL, about mg/mL to about 200 mg/mL, about 50 mg/mL to about 250 mg/mL, about 50 mg/mL to about 300 mg/mL, about 100 mg/mL to about 150 mg/mL, about 100 mg/mL to about 200 mg/mL, about 100 mg/mL to about 250 mg/mL, about 100 mg/mL to about 300 mg/mL, about 150 mg/mL to about 200 mg/mL, about 150 mg/mL to about 250 mg/mL, about 150 mg/mL to about 300 mg/mL, about 200 mg/mL to about 250 mg/mL, about 200 mg/mL to about 300 mg/mL, or about 250 mg/mL to about 300 mg/mL. In some embodiments, the concentration of HPBCD is about 10 mg/mL, about 50 mg/mL, about 100 mg/mL, about 150 mg/mL, about 200 mg/mL, about 250 mg/mL, or about 300 mg/mL. In some embodiments, the concentration of HPBCD is at least about 10 mg/mL, about 50 mg/mL, about 100 mg/mL, about 150 mg/mL, about 200 mg/mL, or about 250 mg/mL. In some embodiments, the concentration of HPBCD is at most about 50 mg/mL, about 100 mg/mL, about 150 mg/mL, about 200 mg/mL, about 250 mg/mL, or about 300 mg/mL.

In some embodiments, the concentration of flumazenil is greater than 0.7 mg/mL. In some embodiments, the concentration of flumazenil is at least 0.8 mg/mL In some embodiments, the concentration of flumazenil is at least 0.9 mg/mL In some embodiments, the concentration of flumazenil is at least 1.0 mg/mL. In some embodiments, the concentration of flumazenil is at least 1.1 mg/mL In some embodiments, the concentration of flumazenil is at least 1.2 mg/mL In some embodiments, the concentration of flumazenil is at least 1.3 mg/mL In some embodiments, the concentration of flumazenil is at least 1.4 mg/mL In some embodiments, the concentration of flumazenil is at least 1.4 mg/mL In some embodiments, the concentration of flumazenil is at least 1.6 mg/mL In some embodiments, the concentration of flumazenil is at least 1.7 mg/mL In some embodiments, the concentration of flumazenil is at least 1.8 mg/mL In some embodiments, the concentration of flumazenil is at least 1.9 mg/mL. In some embodiments, the concentration of flumazenil is at least 2.0 mg/mL. In some embodiments, the concentration of flumazenil is at least 2.2 mg/mL. In some embodiments, the concentration of flumazenil is at least 2.4 mg/mL. In some embodiments, the concentration of flumazenil is at least 2.6 mg/mL. In some embodiments, the concentration of flumazenil is at least 2.8 mg/mL. In some embodiments, the concentration of flumazenil is at least 3.0 mg/mL. In some embodiments, the concentration of flumazenil is at least about 4.0 mg/mL. In some embodiments, the concentration of flumazenil is at least about 5.0 mg/mL.

In some embodiments, the concentration of flumazenil is about 0.8 mg/mL to about 10 mg/mL. In some embodiments, the concentration of flumazenil is about 0.8 mg/mL to about 1 mg/mL, about 0.8 mg/mL to about 1.2 mg/mL, about 0.8 mg/mL to about 1.5 mg/mL, about 0.8 mg/mL to about 1.8 mg/mL, about 0.8 mg/mL to about 2 mg/mL, about 0.8 mg/mL to about 2.5 mg/mL, about 0.8 mg/mL to about 3 mg/mL, about 0.8 mg/mL to about 4 mg/mL, about 0.8 mg/mL to about 5 mg/mL, about 0.8 mg/mL to about 7.5 mg/mL, about 0.8 mg/mL to about 10 mg/mL, about 1 mg/mL to about 1.2 mg/mL, about 1 mg/mL to about 1.5 mg/mL, about 1 mg/mL to about 1.8 mg/mL, about 1 mg/mL to about 2 mg/mL, about 1 mg/mL to about 2.5 mg/mL, about 1 mg/mL to about 3 mg/mL, about 1 mg/mL to about 4 mg/mL, about 1 mg/mL to about 5 mg/mL, about 1 mg/mL to about 7.5 mg/mL, about 1 mg/mL to about 10 mg/mL, about 1.2 mg/mL to about 1.5 mg/mL, about 1.2 mg/mL to about 1.8 mg/mL, about 1.2 mg/mL to about 2 mg/mL, about 1.2 mg/mL to about 2.5 mg/mL, about 1.2 mg/mL to about 3 mg/mL, about 1.2 mg/mL to about 4 mg/mL, about 1.2 mg/mL to about 5 mg/mL, about 1.2 mg/mL to about 7.5 mg/mL, about 1.2 mg/mL to about 10 mg/mL, about 1.5 mg/mL to about 1.8 mg/mL, about 1.5 mg/mL to about 2 mg/mL, about 1.5 mg/mL to about 2.5 mg/mL, about 1.5 mg/mL to about 3 mg/mL, about 1.5 mg/mL to about 4 mg/mL, about 1.5 mg/mL to about 5 mg/mL, about 1.5 mg/mL to about 7.5 mg/mL, about 1.5 mg/mL to about 10 mg/mL, about 1.8 mg/mL to about 2 mg/mL, about 1.8 mg/mL to about 2.5 mg/mL, about 1.8 mg/mL to about 3 mg/mL, about 1.8 mg/mL to about 4 mg/mL, about 1.8 mg/mL to about 5 mg/mL, about 1.8 mg/mL to about 7.5 mg/mL, about 1.8 mg/mL to about 10 mg/mL, about 2 mg/mL to about 2.5 mg/mL, about 2 mg/mL to about 3 mg/mL, about 2 mg/mL to about 4 mg/mL, about 2 mg/mL to about 5 mg/mL, about 2 mg/mL to about 7.5 mg/mL, about 2 mg/mL to about 10 mg/mL, about 2.5 mg/mL to about 3 mg/mL, about 2.5 mg/mL to about 4 mg/mL, about 2.5 mg/mL to about 5 mg/mL, about 2.5 mg/mL to about 7.5 mg/mL, about 2.5 mg/mL to about 10 mg/mL, about 3 mg/mL to about 4 mg/mL, about 3 mg/mL to about 5 mg/mL, about 3 mg/mL to about 7.5 mg/mL, about 3 mg/mL to about 10 mg/mL, about 4 mg/mL to about 5 mg/mL, about 4 mg/mL to about 7.5 mg/mL, about 4 mg/mL to about 10 mg/mL, about 5 mg/mL to about 7.5 mg/mL, about 5 mg/mL to about 10 mg/mL, or about 7.5 mg/mL to about 10 mg/mL. In some embodiments, the concentration of flumazenil is about 0.8 mg/mL, about 1 mg/mL, about 1.2 mg/mL, about 1.5 mg/mL, about 1.8 mg/mL, about 2 mg/mL, about 2.5 mg/mL, about 3 mg/mL, about 4 mg/mL, about 5 mg/mL, about 7.5 mg/mL, or about 10 mg/mL. In some embodiments, the concentration of flumazenil is at least about 0.8 mg/mL, about 1 mg/mL, ab out 1.2 mg/mL, about 1.5 mg/mL, about 1.8 mg/mL, about 2 mg/mL, about 2.5 mg/mL, about 3 mg/mL, about 4 mg/mL, about 5 mg/mL, or about 7.5 mg/mL. In some embodiments, the concentration of flumazenil is at most about 1 mg/mL, about 1.2 mg/mL, about 1.5 mg/mL, about 1.8 mg/mL, about 2 mg/mL, about 2.5 mg/mL, about 3 mg/mL, about 4 mg/mL, about 5 mg/mL, about 7.5 mg/mL, or about 10 mg/mL.

In some embodiments, the concentration of flumazenil is from about 1.0 mg/mL to about 10 mg/mL. In some embodiments, the concentration of flumazenil is from about 1.0 mg/mL to about 8 mg/mL. In some embodiments, the concentration of flumazenil is from about 1.0 mg/mL to about 5 mg/mL. In some embodiments, the concentration of flumazenil is from about 1.5 mg/mL to about 10 mg/mL. In some embodiments, the concentration of flumazenil is from about 1.5 mg/mL to about 8 mg/mL. In some embodiments, the concentration of flumazenil is from about 1.5 mg/mL to about 5 mg/mL. In some embodiments, the concentration of flumazenil is from about 2.0 mg/mL to about 10 mg/mL. In some embodiments, the concentration of flumazenil is from about 2.0 mg/mL to about 8 mg/mL. In some embodiments, the concentration of flumazenil is from about 2.0 mg/mL to about 5 mg/mL. In some embodiments, the concentration of flumazenil is from about 2.5 mg/mL to about 10 mg/mL In some embodiments, the concentration of flumazenil is from about 2.5 mg/mL to about 8 mg/mL. In some embodiments, the concentration of flumazenil is from about 2.5 mg/mL to about 5 mg/mL. In some embodiments, the concentration of flumazenil is from about 3.0 mg/mL to about 10 mg/mL In some embodiments, the concentration of flumazenil is from about 3.0 mg/mL to about 8 mg/mL. In some embodiments, the concentration of flumazenil is from about 3.0 mg/mL to about 5 mg/mL.

In some embodiments, the pH of the pharmaceutical composition is about 4 to about 10. In some embodiments, the pH of the pharmaceutical composition is about 4 to about 5, about 4 to about 6, about 4 to about 7, about 4 to about 8, about 4 to about 9, about 4 to about 10, about 5 to about 6, about 5 to about 7, about 5 to about 8, about 5 to about 9, about 5 to about 10, about 6 to about 7, about 6 to about 8, about 6 to about 9, about 6 to about 10, about 7 to about 8, about 7 to about 9, about 7 to about 10, about 8 to about 9, about 8 to about 10, or about 9 to about 10. In some embodiments, the pH of the pharmaceutical composition is about 4, about 5, about 6, about 7, about 8, about 9, or about 10. In some embodiments, the pH of the pharmaceutical composition is at least about 4, about 5, about 6, or about 7. In some embodiments, the pH of the pharmaceutical composition is at most about 7, about 8, about 9, or about 10.

In some embodiments, the pH of the pharmaceutical composition is about 6 to about 8. In some embodiments, the pH of the pharmaceutical composition is about 6.5 to about 8. In some embodiments, the pH of the pharmaceutical composition is about 7 to about 8. In some embodiments, the pH of the pharmaceutical composition is about 7 to about 8.5. In some embodiments, the pH of the pharmaceutical composition is about 7 to about 9.

In some embodiments, the pH of the pharmaceutical composition is about 5 to about 8. In some embodiments, the pH of the pharmaceutical composition is about 5 to about 7.5. In some embodiments, the pH of the pharmaceutical composition is about 5 to about 7. In some embodiments, the pH of the pharmaceutical composition is about 5 to about 6.5. In some embodiments, the pH of the pharmaceutical composition is about 5 to about 6. In some embodiments, the pH of the pharmaceutical composition is about 5.5 to about 8. In some embodiments, the pH of the pharmaceutical composition is about 5.5 to about 7.5. In some embodiments, the pH of the pharmaceutical composition is about 5.5 to about 7.0. In some embodiments, the pH of the pharmaceutical composition is about 5.5 to about 6.5.

In some embodiments, the osmolality of the pharmaceutical composition is about 250 mOsm/kg to about 500 mOsm/kg. In some embodiments, the osmolality of the pharmaceutical composition is about 250 mOsm/kg to about 275 mOsm/kg, about 250 mOsm/kg to about 300 mOsm/kg, about 250 mOsm/kg to about 325 mOsm/kg, about 250 mOsm/kg to about 350 mOsm/kg, about 250 mOsm/kg to about 400 mOsm/kg, about 250 mOsm/kg to about 500 mOsm/kg, about 275 mOsm/kg to about 300 mOsm/kg, about 275 mOsm/kg to about 325 mOsm/kg, about 275 mOsm/kg to about 350 mOsm/kg, about 275 mOsm/kg to about 400 mOsm/kg, about 275 mOsm/kg to about 500 mOsm/kg, about 300 mOsm/kg to about 325 mOsm/kg, about 300 mOsm/kg to about 350 mOsm/kg, about 300 mOsm/kg to about 400 mOsm/kg, about 300 mOsm/kg to about 500 mOsm/kg, about 325 mOsm/kg to about 350 mOsm/kg, about 325 mOsm/kg to about 400 mOsm/kg, about 325 mOsm/kg to about 500 mOsm/kg, about 350 mOsm/kg to about 400 mOsm/kg, about 350 mOsm/kg to about 500 mOsm/kg, or about 400 mOsm/kg to about 500 mOsm/kg. In some embodiments, the osmolality of the pharmaceutical composition is about 250 mOsm/kg, about 275 mOsm/kg, about 300 mOsm/kg, about 325 mOsm/kg, about 350 mOsm/kg, about 400 mOsm/kg, or about 500 mOsm/kg. In some embodiments, the osmolality of the pharmaceutical composition is at least about 250 mOsm/kg, about 275 mOsm/kg, about 300 mOsm/kg, about 325 mOsm/kg, about 350 mOsm/kg, or about 400 mOsm/kg. In some embodiments, the osmolality of the pharmaceutical composition is at most about 275 mOsm/kg, about 300 mOsm/kg, about 325 mOsm/kg, about 350 mOsm/kg, about 400 mOsm/kg, or about 500 mOsm/kg.

In some embodiments, the osmolality of the pharmaceutical composition is about 300 mOsm/kg. In some embodiments, the osmolality of the pharmaceutical composition is from about 275 to about 300 mOsm/kg. In some embodiments, the osmolality of the pharmaceutical composition is from about 275 to about 325 mOsm/kg. In some embodiments, the osmolality of the pharmaceutical composition is from about 275 to about 350 mOsm/kg. In some embodiments, the osmolality of the pharmaceutical composition is from about 250 to about 300 mOsm/kg. In some embodiments, the osmolality of the pharmaceutical composition is from about 250 to about 325 mOsm/kg. In some embodiments, the osmolality of the pharmaceutical composition is from about 250 to about 350 mOsm/kg.

In some embodiments, the viscosity of the pharmaceutical composition is about 1 centipoise (cP) to about 3 cP. In some embodiments, the viscosity of the pharmaceutical composition is about 1 cP to about 1.5 cP, about 1 cP to about 1.7 cP, about 1 cP to about 1.8 cP, about 1 cP to about 1.9 cP, about 1 cP to about 2 cP, about 1 cP to about 2.1 cP, ab out 1 cP to about 2.2 cP, about 1 cP to about 2.5 cP, about 1 cP to about 3 cP, about 1.5 cP to about 1.7 cP, about 1.5 cP to about 1.8 cP, about 1.5 cP to about 1.9 cP, about 1.5 cP to about 2 cP, about 1.5 cP to about 2.1 cP, about 1.5 cP to about 2.2 cP, about 1.5 cP to about 2.5 cP, about 1.5 cP to about 3 cP, about 1.7 cP to about 1.8 cP, about 1.7 cP to about 1.9 cP, about 1.7 cP to about 2 cP, about 1.7 cP to about 2.1 cP, about 1.7 cP to about 2.2 cP, about 1.7 cP to about 2.5 cP, about 1.7 cP to about 3 cP, about 1.8 cP to about 1.9 cP, about 1.8 cP to about 2 cP, about 1.8 cP to about 2.1 cP, about 1.8 cP to about 2.2 cP, about 1.8 cP to about 2.5 cP, about 1.8 cP to about 3 cP, about 1.9 cP to about 2 cP, about 1.9 cP to about 2.1 cP, about 1.9 cP to about 2.2 cP, about 1.9 cP to about 2.5 cP, about 1.9 cP to about 3 cP, about 2 cP to about 2.1 cP, about 2 cP to about 2.2 cP, about 2 cP to about 2.5 cP, about 2 cP to about 3 cP, about 2.1 cP to about 2.2 cP, about 2.1 cP to about 2.5 cP, about 2.1 cP to about 3 cP, about 2.2 cP to about 2.5 cP, about 2.2 cP to about 3 cP, or about 2.5 cP to about 3 cP. In some embodiments, the viscosity of the pharmaceutical composition is about 1 cP, about 1.5 cP, about 1.7 cP, about 1.8 cP, about 1.9 cP, about 2 cP, about 2.1 cP, about 2.2 cP, about 2.5 cP, or about 3 cP. In some embodiments, the viscosity of the pharmaceutical composition is at least about 1 cP, about 1.5 cP, about 1.7 cP, about 1.8 cP, about 1.9 cP, about 2 cP, about 2.1 cP, about 2.2 cP, or about 2.5 cP. In some embodiments, the viscosity of the pharmaceutical composition is at most about 1.5 cP, about 1.7 cP, about 1.8 cP, about 1.9 cP, about 2 cP, about 2.1 cP, about 2.2 cP, about 2.5 cP, or about 3 cP.

In some embodiments, the viscosity of the pharmaceutical composition is less than about 3 cP. In some embodiments, the viscosity of the pharmaceutical composition is less than about 2.9 cP. In some embodiments, the viscosity of the pharmaceutical composition is less than about 2.8 cP. In some embodiments, the viscosity of the pharmaceutical composition is less than about 2.7 cP In some embodiments, the viscosity of the pharmaceutical composition is less than about 2.6 about cP. In some embodiments, the viscosity of the pharmaceutical composition is less than about 2.5 cP. In some embodiments, the viscosity of the pharmaceutical composition is less than about 2.4 cP. In some embodiments, the viscosity of the pharmaceutical composition is less than 2.3 about cP. In some embodiments, the viscosity of the pharmaceutical composition is less than 2.2 cP. In some embodiments, the viscosity of the pharmaceutical composition is less than about 2.1 cP. In some embodiments, the viscosity of the pharmaceutical composition is less than about 2 cP.

In some embodiments, the pharmaceutical composition comprises a minimal amount of saline. In some embodiments, the pharmaceutical composition comprises a salinity of less than about 0.8% w/v, less than about 0.7% w/v, less than about 0.6% w/v, less than about 0.5% w/v, less than about 0.4% w/v, less than about 0.3% w/v, less than about 0.2% w/v, or less than about 0.1% w/v of sodium chloride.

In some embodiments, the pharmaceutical composition further comprises a preservative. In some embodiments, the preservative is benzethonium chloride. In some embodiments, the benzethonium chloride is present in an amount of about 0.1 mg/mL to about 1 mg/mL. In some embodiments, the preservative is benzethonium chloride, benzalkonium chloride, or chloroxylenol. Other preservatives include benzyl alcohol, methyl parabens, ethyl or n-propyl, and p-hydroxybenzoate. In some embodiments, preservatives are antimicrobial agents, including, but not limited to. Phenol, Meta-cresol, Benzyl alcohol, parabens (methyl, propyl, or butyl), benzalkonium chloride, benzethonium chloride, chlorobutanol, Myristyl gamma picolinium chloride, 2-phenoxyethanol, Phenethyl alcohol, Sorbates (sorbic acid, sodium sorbate), Ethanol, and/or Propylene glycol. In some embodiments, the preservative is present in an amount of about 0.1 mg/mL to about 1 mg/mL. In some embodiments, the preservative is present in an amount of about 0.1 mg/mL to about 0.2 mg/mL, about 0.1 mg/mL to about 0.3 mg/mL, about 0.1 mg/mL to about 0.4 mg/mL, about 0.1 mg/mL to about 0.5 mg/mL, about 0.1 mg/mL to about 0.6 mg/mL, about 0.1 mg/mL to about 0.7 mg/mL, about 0.1 mg/mL to about mg/mL, about 0.1 mg/mL to about 0.9 mg/mL, about 0.1 mg/mL to about 1 mg/mL, about mg/mL to about 0.3 mg/mL, about 0.2 mg/mL to about 0.4 mg/mL, about 0.2 mg/mL to about 0.5 mg/mL, about 0.2 mg/mL to about 0.6 mg/mL, about 0.2 mg/mL to about 0.7 mg/mL, about 0.2 mg/mL to about 0.8 mg/mL, about 0.2 mg/mL to about 0.9 mg/mL, about 0.2 mg/mL to about 1 mg/mL, about 0.3 mg/mL to about 0.4 mg/mL, about 0.3 mg/mL to about 0.5 mg/mL, about 0.3 mg/mL to about 0.6 mg/mL, about 0.3 mg/mL to about 0.7 mg/mL, about 0.3 mg/mL to about 0.8 mg/mL, about 0.3 mg/mL to about 0.9 mg/mL, about 0.3 mg/mL to about 1 mg/mL, about 0.4 mg/mL to about 0.5 mg/mL, about 0.4 mg/mL to about 0.6 mg/mL, about 0.4 mg/mL to about 0.7 mg/mL, about 0.4 mg/mL to about 0.8 mg/mL, about 0.4 mg/mL to about 0.9 mg/mL, about 0.4 mg/mL to about 1 mg/mL, about 0.5 mg/mL to about 0.6 mg/mL, about 0.5 mg/mL to about 0.7 mg/mL, about 0.5 mg/mL to about 0.8 mg/mL, about 0.5 mg/mL to about mg/mL, about 0.5 mg/mL to about 1 mg/mL, about 0.6 mg/mL to about 0.7 mg/mL, about mg/mL to about 0.8 mg/mL, about 0.6 mg/mL to about 0.9 mg/mL, about 0.6 mg/mL to about 1 mg/mL, about 0.7 mg/mL to about 0.8 mg/mL, about 0.7 mg/mL to about 0.9 mg/mL, about 0.7 mg/mL to about 1 mg/mL, about 0.8 mg/mL to about 0.9 mg/mL, about 0.8 mg/mL to about 1 mg/mL, or about 0.9 mg/mL to about 1 mg/mL. In some embodiments, the preservative is present in an amount of about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, or about 1 mg/mL. In some embodiments, the preservative is present in an amount of about at least about 0.1 mg/mL, about 0.2 mg/mL, about 0.3 mg/mL, about 0.4 mg/mL, about mg/mL, about 0.6 mg/mL, about 0.7 mg/mL, about 0.8 mg/mL, or about 0.9 mg/mL. In some embodiments, the preservative is present in an amount of about at most about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, or about 1 mg/mL

In some embodiments, the pharmaceutical composition further comprises a base, a buffer, or a combination thereof.

In some embodiments, the pharmaceutical composition further comprises at least one amino acid, or a functional variant thereof. In some embodiments, the at least one amino acid enhances the solubility of flumazenil. In some embodiments, the at least one amino acid acts as a solubility enhancer for combination use with a complexing agent. In some embodiments, the at least one amino acid includes an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or a functional variant thereof.

In some embodiments, the pharmaceutical composition further comprises an emulsifying agent, a surfactant, a solubilizing agent, a co-solvent or a combination thereof. In some embodiments, the co-solvent is ethanol, propylene glycol, tween 20, tween 80, or glycerin.

In some embodiments, the pharmaceutical composition is adapted to be administered to a subject over a period of time. Such administration can be a continuous dose (e.g. at a constant rate) or can be administered according to a pre-determined schedule (e.g. bolus additions of the pharmaceutical composition over a set schedule over the period of time). In some embodiments, the period of time is at least about 2 days. In some embodiments, the period of time is about 2 days. In some embodiments, the period of time is about 3 days. In some embodiments, the period of time is about 36 hours to about 72 hours. In some embodiments, the period of time is about 36 hours to about 42 hours, about 36 hours to about 48 hours, about 36 hours to about 54 hours, about 36 hours to about 60 hours, about 36 hours to about 66 hours, about 36 hours to about 72 hours, about 42 hours to about 48 hours, about 42 hours to about 54 hours, about 42 hours to about 60 hours, about 42 hours to about 66 hours, about 42 hours to about 72 hours, about 48 hours to about 54 hours, about 48 hours to about 60 hours, about 48 hours to about 66 hours, about 48 hours to about 72 hours, about 54 hours to about 60 hours, about 54 hours to about 66 hours, about 54 hours to about 72 hours, about 60 hours to about 66 hours, about 60 hours to about 72 hours, or about 66 hours to about 72 hours. In some embodiments, the period of time is about 36 hours, about 42 hours, about 48 hours, about 54 hours, about 60 hours, about 66 hours, or about 72 hours. In some embodiments, the period of time is at least about 36 hours, about 42 hours, about 48 hours, about 54 hours, about 60 hours, or about 66 hours. In some embodiments, the period of time is at most about 42 hours, about 48 hours, about 54 hours, about 60 hours, about 66 hours, or about 72 hours.

In some embodiments, the pharmaceutical composition is adapted to deliver a daily dose of at least 0.5 mg of flumazenil. In some embodiments, the pharmaceutical composition is adapted to deliver a daily dose of at least 1 mg of flumazenil. In some embodiments, the pharmaceutical composition is adapted to deliver a daily dose of at least 1.5 mg of flumazenil. In some embodiments, the pharmaceutical composition is adapted to deliver a daily dose of at least 2.0 mg of flumazenil. In some embodiments, the pharmaceutical composition is adapted to deliver a daily dose of at least 2.5 mg of flumazenil. In some embodiments, the pharmaceutical composition is adapted to deliver a daily dose of at least 3.0 mg of flumazenil. In some embodiments, the pharmaceutical composition is adapted to deliver a daily dose of flumazenil of about 0.5 mg to about 4 mg. In some embodiments, the pharmaceutical composition is adapted to deliver a daily dose of flumazenil of about 0.5 mg to about 1 mg, about 0.5 mg to about 1.5 mg, about 0.5 mg to about 2 mg, about 0.5 mg to about 2.5 mg, about 0.5 mg to about 3 mg, about 0.5 mg to about 3.5 mg, about 0.5 mg to about 4 mg, about 1 mg to about 1.5 mg, about 1 mg to about 2 mg, about 1 mg to about 2.5 mg, about 1 mg to about 3 mg, about 1 mg to about 3.5 mg, about 1 mg to about 4 mg, about 1.5 mg to about 2 mg, about 1.5 mg to ab out 2.5 mg, about 1.5 mg to about 3 mg, about 1.5 mg to about 3.5 mg, about 1.5 mg to about 4 mg, about 2 mg to about 2.5 mg, about 2 mg to about 3 mg, about 2 mg to about 3.5 mg, about 2 mg to about 4 mg, about 2.5 mg to about 3 mg, about 2.5 mg to about 3.5 mg, about 2.5 mg to about 4 mg, about 3 mg to about 3.5 mg, about 3 mg to about 4 mg, or about 3.5 mg to about 4 mg. In some embodiments, the pharmaceutical composition is adapted to deliver a daily dose of flumazenil of about 0.5 mg, about 1 mg, about 1.5 mg, about 2 mg, about 2.5 mg, about 3 mg, about 3.5 mg, or about 4 mg. In some embodiments, the pharmaceutical composition is adapted to deliver a daily dose of flumazenil of at least about 0.5 mg, about 1 mg, about 1.5 mg, about 2 mg, about 2.5 mg, about 3 mg, or about 3.5 mg. In some embodiments, the pharmaceutical composition is adapted to deliver a daily dose of flumazenil of at most about 1 mg, about 1.5 mg, about 2 mg, about 2.5 mg, about 3 mg, about 3.5 mg, or about 4 mg. In some embodiments, the pharmaceutical composition is adapted to deliver a daily dose of flumazenil of at least about 4 mg, at least about 4.5 mg, at least about 5 mg, at least about 5.5 mg, or at least about 6 mg. In some embodiments, the pharmaceutical composition is adapted to deliver a daily dose of flumazenil of from about 0.5 mg to about 6 mg. In some embodiments, the pharmaceutical composition is adapted to deliver a daily dose of flumazenil of up to about 10 mg.

In some embodiments, the pharmaceutical composition is adapted to deliver a daily dose of a therapeutically effective amount of the GABA receptor modulator. The daily dose required for the GABA receptor modulator will depend upon the GABA receptor modulator selected based on numerous factors, including its potency, pharmacokinetic properties, and the severity of symptoms of the patient. The daily dose which can be delivered according to the formulations and methods provided herein cam be ascertained by calculations involving the relevant parameters provided herein (e.g. concentration of the GABA receptor modulator in the formulation, the size of the wearable reservoir, etc.). Thus, for such compounds, the daily dose can range from 0.01 mg and lower to upwards of 3000 mg.

In some embodiments, the pharmaceutical composition is adapted to be administered to be delivered by a wearable device, such as a small pump. Such a wearable device should have a sufficiently small footprint to allow the wearer to carry on their daily life with minimal disruption. Thus, the maximum volume of such a device should be kept to a minimum. In some embodiments, the wearable device has a reservoir of the pharmaceutical composition of less than 10 mL. In some embodiments, the wearable device has a reservoir of the pharmaceutical composition of less than 3 mL, 4 mL, 5 mL, 7.5 mL, 10 mL, 12.5 mL, 15 mL, 20 mL, 25 mL or mL. In some embodiments, the wearable device has a reservoir of the pharmaceutical composition of about 2-3 mL, about 2-4 mL, about 2-5 mL, about 2-10 mL, about 3-4 mL, about 3-5 mL, or about 3-10 mL. In some embodiments, the reservoir volume as listed above reflects the maximum volume of the reservoir.

In some embodiments, the pharmaceutical compound is another compound that modulates one or more GABA receptors, such as the GABA_{A} receptor. In some embodiments, the pharmaceutical compound is a GABA_{A} receptor antagonist or modulator, a GABA receptor agonist, a GABA receptor partial agonist, a GABA_{A} receptor negative allosteric modulator or inverse agonist, or salvinorin A, or an isotopic variant thereof; or a pharmaceutically acceptable salt, solvate or hydrate thereof. In some embodiments, the GABA_{A} receptor antagonist or modulator is flumazenil or pentylenetetrazol. In some embodiments, the GABA receptor agonist is muscimol, thiomuscimol, or gaboxadol. In some embodiments, the GABA receptor partial agonist is bretazenil, imidazenil, FG 8205 (7-chloro-5-methyl-3-(5-propan-2-yl-1,2,4-oxadiazol-3-yl)-4H-imidazo[1,5-a][1,4]benzodiazepin-6-one), abecarnil, NS 2710 (1-[1-[3-(3-pyridyl)phenyl]benzimidazol-5-yl]ethanone O-ethyloxime), RWJ-51204 (5-ethoxymethyl-7-fluoro-3-oxo-1,2,3,5-tetrahydrobenzo[4,5] imidazo[1,2a]pyridine-4-N-(2-fluorophenyl)carboxamide), or premazepam. In some embodiments, the GABA_{A} receptor negative allosteric modulator or inverse agonist is bemegride, flurothyl, or pentylenetetrazol. In some embodiments, the a5 subunit containing GABA_{A} receptor selective compound is basmisanil, α5IA (3-(5-methylisoxazol-3-yl)-6-[(1-methyl-1H-1,2,3-triazol-4-yl)methoxy] [1,2,4]triazolo[3,4-a]phthalazine), L-655,708, MRK-016, PWZ-029, a Pyridazine, Ro4938581, TB-21007, FG-7142 (N-Methyl-9H-pyrido[5,4-b]indole-3-carboxamide), Ro16-0154 (ethyl 7-iodanyl-5-methyl-6-oxo-4H-imidazo[1,5-a][1,4]benzodiazepine-3-carboxylate), Radequinil, Ro15-4513 (Ethyl-8-azido-5,6-dihydro-5-methyl-6-oxo-4H-imidazo-1,4-benzodiazepine-3-carboxylate), Sarmazenil, Suritozole, Terbequinil, or ZK-93426 (ethyl-5-isopropoxy-4-methyl-beta-carboline-3-carboxylate). In some embodiments, the GABA receptor modulator is salvinorin A.

The compounds (e.g., GABA receptor modulators (e.g., flumazenil)) of the present disclosure may be in the form of compositions suitable for administration to a subject. In general, such compositions are “pharmaceutical compositions” comprising a compound (e.g., GABA receptor modulators (e.g., flumazenil)) and one or more pharmaceutically acceptable or physiologically acceptable diluents, carriers or excipients. In some embodiments, the compounds (e.g., GABA receptor modulators (e.g., flumazenil)) are present in a therapeutically acceptable amount. The pharmaceutical compositions may be used in the methods of the present disclosure; thus, for example, the pharmaceutical compositions can be administered ex vivo or in vivo to a subject in order to practice the therapeutic and prophylactic methods and uses described herein.

The pharmaceutical compositions of the present disclosure can be formulated to be compatible with the intended method or route of administration; exemplary routes of administration are set forth herein.

In certain embodiments of the pharmaceutical compositions described herein, the co-solvent comprises PEG200, PEG300, PEG400, PEG600, propylene glycol, ethanol, polysorbate 20, polysorbate 80, cremephor, glycerin, benzyl alcohol, dimethylacetamide (DMA), N-methyl-2-pyrrolidone (NMP), tert-butanol, or combinations thereof.

In certain embodiments, the dosage form or pharmaceutical composition comprises a surface-active agent.

In certain embodiments of the pharmaceutical compositions described herein, the surface-active agent comprises polyoxyethylene sorbitan monooleate (Tween 80), sorbitan monooleate, polyoxyethylene sorbitan monolaurate (Tween 20), lecithin, polyoxyethylene-polyoxypropylene copolymers (Pluronics1), or combinations thereof.

In certain embodiments, the dosage form or pharmaceutical composition comprises a non-ionic surfactant.

In certain embodiments of the pharmaceutical compositions described herein, the non-ionic surfactant comprises Cremophor RH40, Cremophor RH60, d-alpha-topopherol polyethylene glycol 1000 succinate, polysorbate 20, polysorbate 80, Solutol HS 15, sorbitan monooleate, poloxamer 407, Labrafil M-1944CS, Labrafil M-2125CS, Labrasol, Gellucire 44/14, Softigen 767, or combinations thereof.

In certain embodiments of the pharmaceutical compositions described herein, the GABA receptor modulator is flumazenil. In certain embodiments of the pharmaceutical compositions described herein, the GABA receptor modulator is a GABA_{A} receptor antagonist or modulator, a GABA receptor agonist, a GABA receptor partial agonist, a GABA_{A} receptor negative allosteric modulator or inverse agonist, or salvinorin A, or an isotopic variant thereof; or a pharmaceutically acceptable salt, solvate or hydrate thereof. In some embodiments, the GABA_{A} receptor antagonist or modulator is flumazenil or pentylenetetrazol. In some embodiments, the GABA receptor agonist is muscimol, thiomuscimol, or gaboxadol. In some embodiments, the GABA receptor partial agonist is bretazenil, imidazenil, FG 8205 (7-chloro-5-methyl-3-(5-propan-2-yl-1,2,4-oxadiazol-3-yl)-4H-imidazo[1,5-a][1,4]benzodiazepin-6-one), abecarnil, NS 2710 (1-[1-[3-(3-pyridyl)phenyl]benzimidazol-5-yl]ethanone O-ethyloxime), RWJ-51204 (5-ethoxymethyl-7-fluoro-3-oxo-1,2,3,5-tetrahydrobenzo[4,5] imidazo[1,2a]pyridine-4-N-(2-fluorophenyl)carboxamide), or premazepam. In some embodiments, the GABA_{A} receptor negative allosteric modulator or inverse agonist is bemegride, flurothyl, or pentylenetetrazol. In some embodiments, the a5 subunit containing GABA_{A} receptor selective compound is basmisanil, α5IA (3-(5-methylisoxazol-3-yl)-6-[(1-methyl-1H-1,2,3-triazol-4-yl)methoxy][1,2,4]triazolo[3,4-a]phthalazine), L-655,708, MRK-016, PWZ-029, a Pyridazine, Ro4938581, TB-21007, FG-7142 (N-Methyl-9H-pyrido[5,4-b]indole-3-carboxamide), Ro16-0154 (ethyl 7-iodanyl-5-methyl-6-oxo-4H-imidazo[1,5-a][1,4]benzodiazepine-3-carboxylate), Radequinil, Ro15-4513 (Ethyl-8-azido-5,6-dihydro-5-methyl-6-oxo-4H-imidazo-1,4-benzodiazepine-3-carboxylate), Sarmazenil, Suritozole, Terbequinil, or ZK-93426 (ethyl-5-isopropoxy-4-methyl-beta-carboline-3-carboxylate). In some embodiments, the GABA receptor modulator is salvinorin A.

In some embodiments, the pharmaceutical composition comprises one or more co-solvents, solubilization/solubilizing agents, stabilization agents, antioxidants, preservatives, cryoprotectants, lyoprotectants, bulking agents, tonicity-adjusting agents, or antimicrobial agents. In some embodiments, the pharmaceutical composition comprises at least one co-solvent. In some embodiments, the pharmaceutical composition comprises at least one solubilizing agent. In some embodiments, the pharmaceutical composition comprises at least one stabilization agent. In some embodiments, the pharmaceutical composition comprises at least one antioxidant. In some embodiments, the pharmaceutical composition comprises at least one preservative. In some embodiments, the pharmaceutical composition comprises at least one cryoprotectant. In some embodiments, the pharmaceutical composition comprises at least one lyoprotectant. In some embodiments, the pharmaceutical composition comprises at least one bulking agent. In some embodiments, the pharmaceutical composition comprises at least one tonicity-adjusting agent. In some embodiments, the pharmaceutical composition comprises at least one antimicrobial agent.

In some embodiments, the formulation or pharmaceutical composition is a pharmaceutical composition. In some embodiments, the formulation is in the form of a sterile injectable aqueous or oleaginous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor® EL (BASF, Parsippany, NJ) or phosphate buffered saline (PBS), ethanol, polyol (e.g., glycerol, propylene glycol, and liquid polyethylene glycol), and suitable mixtures thereof. In addition, sterile fixed oils are conventionally employed as a solvent or suspending medium; for this purpose, any bland fixed oil may be employed, including synthetic mono- or diglycerides. Moreover, fatty acids, such as oleic acid, find use in the preparation of injectables. Prolonged absorption of particular injectable formulations can be achieved by including an agent that delays absorption (e.g., aluminum monostearate or gelatin). In some embodiments, the formulation comprises a co-solvent. In some embodiments, a suitable co-solvent is propylene glycol, glycerin, ethanol, polyethylene glycol (300 and 400), Sorbitol, dimethylacetamide, Cremophor EL, or N-methyl-2-pyrrolidone, or dimethylsulfoxide.

In some embodiments, the formulation or pharmaceutical composition is an aqueous suspension. Aqueous suspensions contain active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives (e.g. benzethonium chloride).

In some embodiments, the formulation or pharmaceutical composition comprises a stabilization agent. In some embodiments, the formulation comprises a surface-active solubilization agent. Surface-active solubilization agents include, but are not limited to. polyoxyethylene sorbitan monooleate (Tween 80), sorbitan monooleate, polyoxyethylene sorbitan monolaurate (Tween 20), lecithin, and Polyoxyethylene-polyoxypropylene copolymers (Pluronics1). In some embodiments, the formulation comprises a non-ionic surfactant solubilization agent. Non-ionic surfactants include, but are not limited. Cremophor RH 40, Cremophor RH 60, d-alpha-tocopherol polyethylene glycol 1000 succinate, polysorbate 20, polysorbate 80, Solutol HS 1, sorbitan monooleate, poloxamer 407, Labrafil M-1944CS, Labrafil M-2125 CS, Labrasol, Gellucire 44/14, Softigen 767, and mono-fatty esters and di-fatty acid esters of PEG 300, 400, and 1750. In some embodiments, the formulation comprises a phospholipid solubilizing agent such as, hydrogenated soy phosphatidylcholine, phosphatidylcholine, distearoylphosphatidylglycerol, L-alpha-dimyristoylphosphatidylcholine, or L-alpha-dimyristoylphosphatidylglycerol.

In some embodiments, the formulation or pharmaceutical composition comprises a complexation agent. In some embodiments, the complexation agent is hydroxypropyl-b-cyclodextrin, bulfobutylether-b-cyclodextrin (Captisol1), or polyvinylpyrrolidone. In some embodiments, the complexation agent is an amino acid such as, arginine, lysine, or histidine. In some embodiments, the formulation or pharmaceutical composition comprises a cyclodextrin excipient. Cyclodextrin excipients are used to enhance the stability, tolerability and absorption of compounds in parenteral aqueous solutions. Common cyclodextrin excipients include but are not limited to. alpha-Cyclodextrin (alpha-CD), beta-Cyclodextrin (beta-CD), gamma-Cyclodextrin (gamma-CD), Diethyl-ethyl-beta-cyclodextrin (DE-beta-CD), Dimethyl-ethyl-beta-cyclodextrin (DM-beta-CD), Hydroxypropyl-beta-cyclodextrin (HP-beta-CD), Hydroxypropyl-gamma-cyclodextrin (HP-gamma-CD), Methyl-b-cyclodextrin (M-b eta-CD), Sulfobutylether-beta-cyclodextrin (SBE-beta-CD), Randomly methylated-beta-CD (RM-beta-CD), Maltosyl-beta-CD (mal-beta-CD), Hydroxypropyl-alpha-CD.

The formulations or pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The formulation or pharmaceutical composition typically comprises a therapeutically effective amount of an active compound (e.g. a GABA receptor modulator, such as flumazenil), or a hydrate, solvate, tautomer, or pharmaceutically acceptable salt thereof, and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate-buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a triethanolamine (Tris) buffer, histidine, bicarbonate; N-(2-Hydroxyethyl)piperazine-N′-(2-ethanesulfonic acid) (HEPES); 2-(N-Morpholino)ethanesulfonic acid (MES); 2-(N-Morpholino)ethanesulfonic acid sodium salt (MES); 3-(N-Morpholino)propanesulfonic acid (MOPS); and N-tris[Hydroxymethyl]methyl-3-aminopropanesulfonic acid (TAPS).

Many active pharmaceutical ingredients (APIs) are weak acids or weak bases. Weak acids or weak bases can exist in an un-ionized form or as an ionized complex prepared by the addition of a base or acid respectively. The resultant complex is stabilized by ionic interactions and is known as a salt. This complex exists via an ionic bond between an ionized API and an oppositely charged counterion. Salts offer a number of advantages over their un-ionized counterparts. The choice of counterion can have a large influence on the salts properties and the use of a given salt form of a given API in a pharmaceutical product is influenced and guided by a number of factors for example stability (photo, hydrolytic and thermal), solubility, physicochemical properties, solid state properties (crystallinity, polymorphism, particle size, crystal morphology, melting point, compactability), production considerations (e.g., ease of handling and processing), dissolution rate, modulation of drug release, compatibility with excipients and containers, ease and consistency of production, desired route of administration, and organoleptic factors (e.g., taste). Furthermore, with respect to injection, salt can influence pain and irritation at the injection site.

With regard to cyclodextrin solubilization, specific salts of various APIs have been found to form multicomponent complexes/systems or ternary systems which can have distinct desirable properties as compared to their standard binary complexes/systems counterparts prepared between the cyclodextrin and the un-ionized API, as well as compared to other multicomponent ternary complexes/systems involving different salt forms of that API. These multicomponent complexes/systems can thus dramatically influence solubility of the API in aqueous solutions, dissolution rates, can influence product stability, and pharmacokinetic properties of the pharmaceutical preparation.

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampule, syringe, or autoinjector (similar to, e.g., an EpiPen®)), whereas a multi-use container (e.g., a multi-use vial) is provided in other embodiments.

Formulations or pharmaceutical compositions can also include carriers to protect the composition against rapid degradation or elimination from the body, such as a controlled release formulation, including liposomes, hydrogels, prodrugs and microencapsulated delivery systems. For example, a time-delay material such as glyceryl monostearate or glyceryl stearate alone, or in combination with a wax, may be employed. Any drug delivery apparatus may be used to deliver a GABA receptor modulator, including implants (e.g., implantable pumps) and catheter systems, slow injection pumps and devices, all of which are well known to the skilled artisan.

In some embodiments, the formulation or pharmaceutical composition is stored in a reservoir of the drug delivery device. In some embodiments, the formulation is stored in a cartridge that is insertable and/or attachable to the drug delivery device. In some embodiments, the cartridge and/or drug delivery device comprises a product label for intramuscular injection. In some embodiments, the cartridge and/or drug delivery device comprises a product label for subcutaneous injection. In some embodiments, the cartridge and/or drug delivery device comprises a product label for intravenous injection. In some embodiments, disclosed herein is a kit comprising a product label for intramuscular injection. In some embodiments, disclosed herein is a kit comprising a product label for subcutaneous injection. In some embodiments, disclosed herein is a kit comprising a product label for intravenous injection.

In some embodiments, the formulation or pharmaceutical composition is a liquid formulation comprising flumazenil. In some embodiments, the flumazenil is at least about 90%, about 95%, about 96%, about 97%, about 98%, or about 99% pure. In some embodiments, the flumazenil is at least about 99.1%, about 99.2%, about 99.3%, about 99.4%, about 99.5%, about 99.6%, about 99.8%, or about 99.9% pure. In some embodiments, the flumazenil comprises less than about 5%, about 4%, about 3%, about 2%, or about 1% impurities.

It is frequently beneficial to improve one of more physical properties of the treatment modalities disclosed herein and/or the manner in which they are administered. Improvements of physical properties include, for example, methods of increasing water solubility, bioavailability, serum half-life, and/or therapeutic half-life; and/or modulating biological activity. Modifications known in the art include pegylation, Fc-fusion and albumin fusion. Although generally associated with large molecule agents (e.g., polypeptides), such modifications have recently been evaluated with particular small molecules. By way of example, Chiang, M. et al. (J. Am. Chem. Soc., 2014, 136(9).3370-73) describe a small molecule agonist of the adenosine 2a receptor conjugated to the immunoglobulin Fc domain. The small molecule-Fc conjugate retained potent Fc receptor and adenosine 2a receptor interactions and showed superior properties compared to the unconjugated small molecule. Covalent attachment of PEG molecules to small molecule therapeutics has also been described (Li, W. et al., Progress in Polymer Science, 2013 38.421-44).

The GABA receptor modulator of the present disclosure may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED₅₀ of an agent is the dose or amount of an agent that produces a therapeutic response or desired effect in 50% of the population to which it is administered. Although the ED 50 is commonly used as a measure of reasonable expectance of an agent's effect, it is not necessarily the dose that a clinician might deem appropriate taking into consideration all relevant factors. Thus, in some situations the effective amount is more than the calculated ED₅₀, in other situations the effective amount is less than the calculated ED₅₀, and in still other situations the effective amount is the same as the calculated ED₅₀.

In addition, an effective dose of the GABA receptor modulator (e.g. flumazenil of the present disclosure may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In embodiments, the dosage of the compound is contained in a “unit dosage form.” The phrase “unit dosage form” refers to physically discrete units, each unit including a predetermined amount of the compound (e.g., flumazenil, ora hydrate, solvate, or pharmaceutically acceptable salt thereof), sufficient to produce the desired effect. It will be appreciated that the parameters of a unit dosage form will depend on the particular agent and the effect to be achieved.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents, such as those set forth above, and flavoring agents may be added to provide a palatable oral preparation.

Dispersible powders and granules suitable for preparation of an aqueous suspension by the addition of water provide the active ingredient in admixture with a dispersing or wetting agent, and optionally one or more suspending agents and/or preservatives. Suitable dispersing or wetting agents and suspending agents are exemplified herein.

Depot injections, which are generally administered subcutaneously or intramuscularly, may also be utilized to release the compound (e.g., GABA receptor modulator (e.g., flumazenil)) disclosed herein over a defined period of time. Depot injections are usually either solid- or oil-based and generally comprise at least one of the formulation components set forth herein. One of ordinary skill in the art is familiar with possible formulations and uses of depot injections.

Some formulations include one or more stabilization agents. Potential stabilization agents that are contemplated include buffers. Acetate, Citrate, Sodium Citrate, Tartrate, Phosphate, histidine, bicarbonate, Triethanolamine (TRIS) and their salts. In some formulations, the potential stabilization agents might include antioxidants and preservatives such as. Ascorbic acid, Acetylcysteine (NAC), Sulfurous acid salts (bisulfate, metabisulfite), Monothioglyercol. Butylated hydroxyanisole (BHA), Butylated hydroxytoluene (BHT), Tert-butylhydroquinone (TBHQ), 2′,4′,5′-Trihydroxybutyrophenone phenylhydrazone (THBP), Ethylenediaminetetraacetic acid (EDTA), Sodium formaldehyde sulfoxylate (SFS), Tocopherol (Vitamin E), Ascorbyl palmitate, Gallates (e.g., propyl gallate, octyl gallate, lauryl gallate), Cysteine ethyl ether, Tartaric acid, Phosphoric acid, Thiourea, Sodium thioglycolate, Nitrogen, and/or Argon.

In some formulations, the potential stabilization agents might include bulking agents, cryoprotectants, and lyoprotectants. Agents that were considered include. Mannitol, Glycine, Sucrose, Lactose, Trehalose, Dextran, Povidone, Sorbitol and/or Polydextrose. In some formulations potential stabilization agents might include tonicity-adjusting agents. Agents that were considered include. sodium chloride, Glycerin, Mannitol, Dextrose, and/or glycerol. In some formulations the potential stabilization agents might include antimicrobial agents including, but not limited to. Phenol, Meta-cresol, Benzyl alcohol, parabens (methyl, propyl, or butyl), benzalkonium chloride, benzethonium chloride, chlorobutanol, Myristyl gamma picolinium chloride, 2-phenoxyethanol, Phenethyl alcohol, Sorbates (sorbic acid, sodium sorbate), Ethanol, and/or Propylene glycol.

In some formulations, soothing agents might include topical analgesics such as. lidocaine, benzocaine, tetracaine, bupivicaine, ropivacaine, and/or levobupivacaine.

In some formulations, emulsion stabilizers include hydroxyethyl cellulose, hydroxypropylcellulose, and/or hydroxypropyl methyl cellulose (hypromellose).

The compound (e.g., GABA receptor modulator (e.g., flumazenil)) contemplated by the present disclosure may be in the form of any other suitable pharmaceutical composition currently known or developed in the future.

### III. Methods

In an aspect, provided herein, is a method of treating a disease or disorder in a subject in need thereof, comprising administering to a subject in need thereof a therapeutically effective amount of flumazenil. The flumazenil may be administered as any of the pharmaceutical compositions provided herein. In some embodiments, the flumazenil is administered by subcutaneous injection in an amount of at least 0.5 mg/day from a single device for a period of time of at least 2 days, wherein the single device has a reservoir volume of less than 30 mL.

Also provided herein is a method of treating a disease or disorder in a subject in need thereof, comprising administering to a subject in need thereof a therapeutically effective amount of a pharmaceutical compound, wherein the pharmaceutical compound is a GABA_{A} receptor antagonist or modulator, a GABA receptor agonist, a GABA receptor partial agonist, a GABA_{A} receptor negative allosteric modulator or inverse agonist, or salvinorin A, wherein the flumazenil is administered by subcutaneous injection from a single device fora period of time of at least 2 days, wherein the single device has a reservoir volume less than 30 mL.

In some embodiments, the disease or disorder is benzodiazepine dependence, withdrawal or toxicity. In some embodiments, the disease or disorder is benzodiazepine withdrawal. In some embodiments, the benzodiazepine withdrawal is acute or post-acute withdrawal, or a combination thereof. In some embodiments, the benzodiazepine withdrawal is acute withdrawal. In some embodiments, the benzodiazepine withdrawal is post-acute withdrawal. In some embodiments, the benzodiazepine withdrawal is both acute and post-acute withdrawal.

In some embodiments, the disease or disorder is alcohol dependence, withdrawal, or toxicity, sedative dependence, withdrawal, or toxicity, hypnotic dependence, withdrawal, or toxicity, anxiolytic dependence, withdrawal, or toxicity, panic disorder, generalized anxiety disorder, post-traumatic stress disorder (PTSD), idiopathic hypersomnia, narcolepsy, mood disorders, major depression, dysthymia, chronic suicidality, anxiety disorder NOS, obsessive compulsive disorder, eating disorder NOS, anorexia nervosa, bulimia nervosa, intermittent explosive disorder, a sleep disorder, insomnia NOS, a pain disorder, or a chronic pain disorder.

In some embodiments, the single device is adapted to administer the flumazenil or other pharmaceutical compound to the subject over a period of time. Such administration can be a continuous dose (e.g. at a constant rate) or can be administered according to a pre-determined schedule (e.g. bolus additions of the pharmaceutical composition over a set schedule over the period of time). In some embodiments, the period of time is at least about 2 days. In some embodiments, the period of time is about 2 days. In some embodiments, the period of time is about 3 days. In some embodiments, the period of time is about 36 hours to about 72 hours. In some embodiments, the period of time is about 36 hours to about 42 hours, about 36 hours to about 48 hours, about 36 hours to about 54 hours, about 36 hours to about 60 hours, about 36 hours to about 66 hours, about 36 hours to about 72 hours, about 42 hours to about 48 hours, about 42 hours to about 54 hours, about 42 hours to about 60 hours, about 42 hours to about 66 hours, about 42 hours to about 72 hours, about 48 hours to about 54 hours, about 48 hours to about 60 hours, about 48 hours to about 66 hours, about 48 hours to about 72 hours, about 54 hours to about 60 hours, about 54 hours to about 66 hours, about 54 hours to about 72 hours, about 60 hours to about 66 hours, about 60 hours to about 72 hours, or about 66 hours to about 72 hours. In some embodiments, the period of time is about 36 hours, about 42 hours, about 48 hours, about 54 hours, about 60 hours, about 66 hours, or about 72 hours. In some embodiments, the period of time is at least about 36 hours, about 42 hours, about 48 hours, about 54 hours, about 60 hours, or about 66 hours. In some embodiments, the period of time is at most about 42 hours, about 48 hours, about 54 hours, about 60 hours, about 66 hours, or about 72 hours.

In some embodiments, the single device is adapted to deliver a daily dose of at least 0.5 mg of flumazenil. In some embodiments, the pharmaceutical composition is adapted to deliver a daily dose of at least 1 mg of flumazenil. In some embodiments, the single device is adapted to deliver a daily dose of at least 1.5 mg of flumazenil. In some embodiments, the single device is adapted to deliver a daily dose of at least 2.0 mg of flumazenil. In some embodiments, the single device is adapted to deliver a daily dose of at least 2.5 mg of flumazenil. In some embodiments, the single device is adapted to deliver a daily dose of at least 3.0 mg of flumazenil. In some embodiments, the single device is adapted to deliver a daily dose of flumazenil of about 0.5 mg to about 4 mg. In some embodiments, the single device is adapted to deliver a daily dose of flumazenil of about 0.5 mg to about 1 mg, about 0.5 mg to about 1.5 mg, about 0.5 mg to about 2 mg, about 0.5 mg to about 2.5 mg, about 0.5 mg to about 3 mg, about 0.5 mg to about 3.5 mg, about 0.5 mg to about 4 mg, about 1 mg to about 1.5 mg, about 1 mg to about 2 mg, about 1 mg to about 2.5 mg, about 1 mg to about 3 mg, about 1 mg to about 3.5 mg, about 1 mg to about 4 mg, about 1.5 mg to about 2 mg, about 1.5 mg to about 2.5 mg, about 1.5 mg to about 3 mg, about 1.5 mg to about 3.5 mg, about 1.5 mg to about 4 mg, about 2 mg to about 2.5 mg, about 2 mg to about 3 mg, about 2 mg to about 3.5 mg, about 2 mg to about 4 mg, about 2.5 mg to about 3 mg, about 2.5 mg to about 3.5 mg, about 2.5 mg to about 4 mg, about 3 mg to about 3.5 mg, about 3 mg to about 4 mg, or about 3.5 mg to about 4 mg. In some embodiments, the single device is adapted to deliver a daily dose of flumazenil of about 0.5 mg, about 1 mg, about 1.5 mg, about 2 mg, about 2.5 mg, about 3 mg, about 3.5 mg, or about 4 mg. In some embodiments, single device is adapted to deliver a daily dose of flumazenil of at least about 0.5 mg, about 1 mg, about 1.5 mg, about 2 mg, about 2.5 mg, about 3 mg, or about 3.5 mg. In some embodiments, the single device is adapted to deliver a daily dose of flumazenil of at most about 1 mg, about 1.5 mg, about 2 mg, about 2.5 mg, about 3 mg, about 3.5 mg, or about 4 mg. In some embodiments, the pharmaceutical composition is adapted to deliver a daily dose of flumazenil of up to about 10 mg. In some embodiments, the flumazenil is administered in an amount of at least 0.5 mg/day, at least 1.0 mg/day, at least 1.5 mg/day, at least 2 mg/day, at least 3 mg/day, at least 4 mg/day, at least 5 mg/day, at least 7.5 mg/day, or at least 10 mg/day.

In some embodiments, the pharmaceutical composition is adapted to be administered to be delivered by a wearable device, such as a small pump. Such a wearable device should have a sufficiently small footprint to allow the wearer to carry on their daily life with minimal disruption. Thus, the maximum volume of such a device should be kept to a minimum. In some embodiments, is administered by a wearable mini-pump. In some embodiments, the wearable mini-pump is a 2-day pump. In some embodiments, the wearable mini-pump is a 3-day pump. In some embodiments, a single wearable mini-pump is used. In some embodiments, the single device has a reservoir volume of about 2 mL to about 25 mL. In some embodiments, the single device has a reservoir volume of about 2 mL to about 3 mL, about 2 mL to about 5 mL, about 2 mL to about 7.5 mL, about 2 mL to about 10 mL, about 2 mL to about 12.5 mL, about 2 mL to about 15 mL, about 2 mL to about 20 mL, about 2 mL to about 25 mL, about 3 mL to about 5 mL, about 3 mL to about 7.5 mL, about 3 mL to about 10 mL, about 3 mL to about 12.5 mL, about 3 mL to about 15 mL, about 3 mL to about 20 mL, about 3 mL to about 25 mL. In some embodiments, the single device has a reservoir volume of at most about 3 mL, about 5 mL, about 7.5 mL, about 10 mL, about 12.5 mL, about 15 mL, about 20 mL, or about 25 mL. In some embodiments, the reservoir volume is less than 20 mL. in some embodiments, the reservoir volume is less than 15 mL. In some embodiments, the reservoir volume is less than 10 mL. In some embodiments, the reservoir volume is less than 5 mL. In some embodiments, the reservoir volume is less than 4 mL. In some embodiments, the reservoir volume is less than 3.5 mL. In some embodiments, the reservoir volume is less than 3 mL. In some embodiments, the reservoir volume as listed above reflects the maximum volume of the reservoir of the single device.

In some embodiments, the flumazenil is administered as a pharmaceutical composition having a concentration of flumazenil of greater than 0.7 mg/mL. In some embodiments, the concentration of flumazenil is about 0.8 mg/mL to about 10 mg/mL. In some embodiments, the concentration of flumazenil is about 0.8 mg/mL to about 1 mg/mL, about 0.8 mg/mL to about 1.2 mg/mL, about 0.8 mg/mL to about 1.5 mg/mL, about 0.8 mg/mL to about 1.8 mg/mL, about 0.8 mg/mL to about 2 mg/mL, about 0.8 mg/mL to about 2.5 mg/mL, about 0.8 mg/mL to about 3 mg/mL, about 0.8 mg/mL to about 4 mg/mL, about 0.8 mg/mL to about 5 mg/mL, about 0.8 mg/mL to about 7.5 mg/mL, about 0.8 mg/mL to about 10 mg/mL, about 1 mg/mL to about 1.2 mg/mL, about 1 mg/mL to about 1.5 mg/mL, about 1 mg/mL to about 1.8 mg/mL, about 1 mg/mL to about 2 mg/mL, about 1 mg/mL to about 2.5 mg/mL, about 1 mg/mL to about 3 mg/mL, about 1 mg/mL to about 4 mg/mL, about 1 mg/mL to about 5 mg/mL, about 1 mg/mL to about 7.5 mg/mL, about 1 mg/mL to about 10 mg/mL, about 1.2 mg/mL to about 1.5 mg/mL, about 1.2 mg/mL to about 1.8 mg/mL, about 1.2 mg/mL to about 2 mg/mL, about 1.2 mg/mL to about 2.5 mg/mL, about 1.2 mg/mL to about 3 mg/mL, about 1.2 mg/mL to about 4 mg/mL, about 1.2 mg/mL to about 5 mg/mL, about 1.2 mg/mL to about 7.5 mg/mL, about 1.2 mg/mL to about 10 mg/mL, about 1.5 mg/mL to about 1.8 mg/mL, about 1.5 mg/mL to about 2 mg/mL, about 1.5 mg/mL to about 2.5 mg/mL, about 1.5 mg/mL to about 3 mg/mL, about 1.5 mg/mL to about 4 mg/mL, about 1.5 mg/mL to about 5 mg/mL, about 1.5 mg/mL to about 7.5 mg/mL, about 1.5 mg/mL to about 10 mg/mL, about 1.8 mg/mL to about 2 mg/mL, about 1.8 mg/mL to about 2.5 mg/mL, about 1.8 mg/mL to about 3 mg/mL, about 1.8 mg/mL to about 4 mg/mL, about 1.8 mg/mL to about 5 mg/mL, about 1.8 mg/mL to about 7.5 mg/mL, about 1.8 mg/mL to about 10 mg/mL, about 2 mg/mL to about 2.5 mg/mL, about 2 mg/mL to about 3 mg/mL, about 2 mg/mL to about 4 mg/mL, about 2 mg/mL to about 5 mg/mL, about 2 mg/mL to about 7.5 mg/mL, about 2 mg/mL to about 10 mg/mL, about 2.5 mg/mL to about 3 mg/mL, about 2.5 mg/mL to about 4 mg/mL, about 2.5 mg/mL to about 5 mg/mL, about 2.5 mg/mL to about 7.5 mg/mL, about 2.5 mg/mL to about 10 mg/mL, about 3 mg/mL to about 4 mg/mL, about 3 mg/mL to about 5 mg/mL, about 3 mg/mL to about 7.5 mg/mL, about 3 mg/mL to about 10 mg/mL, about 4 mg/mL to about 5 mg/mL, about 4 mg/mL to about 7.5 mg/mL, about 4 mg/mL to about 10 mg/mL, about 5 mg/mL to about 7.5 mg/mL, about 5 mg/mL to about 10 mg/mL, or about 7.5 mg/mL to about 10 mg/mL. In some embodiments, the concentration of flumazenil is about 0.8 mg/mL, about 1 mg/mL, about 1.2 mg/mL, about 1.5 mg/mL, about 1.8 mg/mL, about 2 mg/mL, about 2.5 mg/mL, about 3 mg/mL, about 4 mg/mL, about 5 mg/mL, about 7.5 mg/mL, or about 10 mg/mL. In some embodiments, the concentration of flumazenil is at least about 0.8 mg/mL, about 1 mg/mL, about 1.2 mg/mL, about 1.5 mg/mL, about 1.8 mg/mL, about 2 mg/mL, about 2.5 mg/mL, about 3 mg/mL, about 4 mg/mL, about 5 mg/mL, or about 7.5 mg/mL. In some embodiments, the concentration of flumazenil is at most about 1 mg/mL, about 1.2 mg/mL, about 1.5 mg/mL, about 1.8 mg/mL, about 2 mg/mL, about 2.5 mg/mL, about 3 mg/mL, about 4 mg/mL, about 5 mg/mL, about 7.5 mg/mL, or about 10 mg/mL.

In some embodiments, the flumazenil or other pharmaceutical compound is administered at a specified rate. Such rate can be continuous, or can be the rate of a particular bolus injection of the compound. In some embodiments, the rate is an average rate over a 24 hour period. In some embodiments, the flumazenil is administered at a rate of about 40 ug/hr to about 250 μg/hr. In some embodiments, the flumazenil is administered at a rate of about 40 μg/hr to about μg/hr, about 40 μg/hr/hr to about 80 μg/hr, about 40 μg/hr to about 100 μg/hr, about 40 μg/hr to about 150 μg/hr, about 40 μg/hr to about 200 μg/hr, about 40 μg/hr to about 250 μg/hr, about 60 μg/hr to about 80 μg/hr, about 60 μg/hr to about 100 μg/hr, about 60 μg/hr to about 150 μg/hr, about 60 μg/hr to about 200 μg/hr, about 60 μg/hr to about 250 μg/hr, about 80 μg/hr to about 100 μg/hr, about 80 μg/hr to about 150 μg/hr, about 80 μg/hr to about 200 μg/hr, about μg/hr to about 250 μg/hr, about 100 μg/hr to about 150 μg/hr, about 100 μg/hr to about 200 μg/hr, about 100 μg/hr to about 250 μg/hr, about 150 μg/hr to about 200 μg/hr, about 150 μg/hr to about 250 μg/hr, or about 200 μg/hr to about 250 μg/hr. In some embodiments, the flumazenil is administered at a rate of about μg/hr, about 60 μg/hr, about 80 μg/hr, about 100 μg/hr, about 150 μg/hr, about 200 μg/hr, or about 250 μg/hr. In some embodiments, the flumazenil is administered at a rate of at least about 40 μg/hr, about 60 μg/hr, about 80 μg/hr, about 100 μg/hr, about 150 μg/hr, or about 200 μg/hr. In some embodiments, the flumazenil is administered at a rate of at most about 60 μg/hr, about 80 μg/hr, about 100 μg/hr, about 150 μg/hr, about 200 μg/hr, or about 250 μg/hr.

In some embodiments, the pharmaceutical compound is administered at a rate of about μg/hr to about 250 μg/hr. In some embodiments, the pharmaceutical compound is administered at a rate of about 40 μg/hr to about 60 μg/hr, about 40 μg/hr to about 80 μg/hr, about 40 μg/hr to about 100 μg/hr, about 40 μg/hr to about 150 μg/hr, about 40 μg/hr to about 200 μg/hr, about 40 μg/hr, to about 250 μg/hr, about 60 μg/hr to about 80 μg/hr, about 60 μg/hr to about 100 μg/hr, about 60 μg/hr to about 150 μg/hr, about 60 μg/hr to about 200 μg/hr, about μg/hr to about 250 μg/hr, about 80 μg/hr to about 100 μg/hr, about 80 μg/hr to about 150 μg/hr, about 80 μg/hr r to about 200 μg/hr, about 80 μg/hr to about 250 μg/hr, about 100 μg/hr to about 150 μg/hr, about 100 μg/hr to about 200 μg/hr, about 100 μg/hr to about 250 μg/hr, about 150 μg/hr to about 200 μg/hr, about 150 μg/hr to about 250 μg/hr, or about 200 μg/hr to about 250 μg/hr. In some embodiments, the pharmaceutical compound is administered at a rate of about 40 μg/hr, about 60 μg/hr, about 80 μg/hr, about 100 μg/hr, about 150 μg/hr, about 200 μg/hr, or about 250 μg/hr. In some embodiments, the pharmaceutical compound is administered at a rate of at least about 40 μg/hr, about 60 μg/hr, about 80 μg/hr, about 100 μg/hr, about 150 μg/hr, or about 200 μg/hr. In some embodiments, the pharmaceutical compound is administered at a rate of at most about 60 μg/hr, about 80 μg/hr, about 100 μg/hr, about 150 μg/hr, about 200 μg/hr, or about 250 μg/hr.

In some embodiments, the rate of administration may vary over the course of the dosage regimen. For example, an initial rate of administration may be slower during a treatment for benzodiazepine withdrawal when the subject is undergoing a benzodiazepine taper. Additionally, the rate of administration may lower at the end of the regimen in order to curtail any potential side effects or withdrawal symptoms from administration of the pharmaceutical compound (e.g. flumazenil) itself.

For treatment regimens of certain diseases or disorders, it may be necessary to administer flumazenil or other pharmaceutical compound over a longer period of time than can be accommodated by a single small reservoir device (e.g., a treatment for benzodiazepine dependence or withdrawal, which may require an administration of flumazenil over a period of up to 30 days or more). Thus, in some embodiments, a subject is administered the course of the therapy by using several devices comprising a reservoir of flumazenil or other pharmaceutical compound in a sequential manner. Thus, in some embodiments, the method further comprises administering to the subject a therapeutically effective amount of flumazenil or other pharmaceutical compound from one or more additional single devices, wherein each additional single device is administered to the subject after an additional iteration of the period of time. In some embodiments, the course of treatment requires administration of flumazenil or other pharmaceutical compound from at least 2, at least 3, at least 4, at least 5, at least 6, at least 7, at least 8, at least 9, or at least 10 or more devices. Each device may be used for the same or a different amount of time depending on the duration and course of the therapy. Additionally, the concentration of API in each device and the rate of administration may also be the same or varied across devices.

In some embodiments, the total time of administration (e.g., administration across multiple devices) is at least 5 days, at least 10 days, at least 15 days, at least 20 days, at least 25 days, or at least 30 days. In some embodiments, the total time of administration is about 5 days to about 30 days. In some embodiments, the total time of administration is about 5 days to about days, about 5 days to about 15 days, about 5 days to about 20 days, about 5 days to about 25 days, about 5 days to about 30 days, about 10 days to about 15 days, about 10 days to about 20 days, about 10 days to about 25 days, about 10 days to about 30 days, about 15 days to about 20 days, about 15 days to about 25 days, about 15 days to ab out 30 days, about 20 days to about 25 days, about 20 days to about 30 days, or about 25 days to about 30 days. In some embodiments, the total time of administration is about 5 days, about 10 days, about 15 days, about 20 days, about 25 days, or about 30 days. In some embodiments, the total time of administration is at least about 5 days, about 10 days, about 15 days, about 20 days, or about 25 days. In some embodiments, the total time of administration is at most about 10 days, about 15 days, about 20 days, about 25 days, or about 30 days. In some embodiments, the total time of administration is about 5 days to about 90 days. In some embodiments, the total time of administration is about 5 days to about 15 days, about 5 days to about 30 days, about 5 days to about 45 days, about 5 days to about 60 days, about 5 days to about 75 days, about 5 days to about 90 days, about 15 days to about 30 days, about 15 days to about 45 days, about 15 days to about 60 days, about 15 days to about 75 days, about 15 days to about 90 days, about 30 days to about 45 days, about 30 days to about 60 days, about 30 days to about 75 days, about 30 days to about 90 days, about 45 days to about 60 days, about 45 days to about 75 days, about 45 days to about 90 days, about 60 days to about 75 days, about 60 days to about 90 days, or about 75 days to about 90 days. In some embodiments, the total time of administration is about 5 days, about 15 days, about 30 days, about 45 days, about 60 days, about 75 days, or about 90 days. In some embodiments, the total time of administration is at least about 5 days, about 15 days, about 30 days, about 45 days, about 60 days, or about 75 days. In some embodiments, the total time of administration is at most about 15 days, about 30 days, about 45 days, about 60 days, about 75 days, or about 90 days.

In some embodiments, the pharmaceutical compound is a compound that modulates one or more GABA receptors, such as the GABA_{A} receptor. In some embodiments, the pharmaceutical compound is a GABA_{A} receptor antagonist or modulator, a GABA receptor agonist, a GABA receptor partial agonist, a GABA_{A} receptor negative allosteric modulator or inverse agonist, or salvinorin A, or an isotopic variant thereof; or a pharmaceutically acceptable salt, solvate or hydrate thereof. In some embodiments, the GABA_{A} receptor antagonist or modulator is flumazenil or pentylenetetrazol. In some embodiments, the GABA receptor agonist is muscimol, thiomuscimol, or gaboxadol. In some embodiments, the GABA receptor partial agonist is bretazenil, imidazenil, FG 8205 (7-chloro-5-methyl-3-(5-propan-2-yl-1,2,4-oxadiazol-3-yl)-4H-imidazo[1,5-a][1,4]benzodiazepin-6-one), abecarnil, NS 2710 (1-[1-[3-(3-pyridyl)phenyl]benzimidazol-5-yl]ethanone O-ethyloxime), RWJ-51204 (5-ethoxymethyl-7-fluoro-3-oxo-1,2,3,5-tetrahydrobenzo[4,5] imidazo[1,2a]pyridine-4-N-(2-fluorophenyl)carboxamide), or premazepam. In some embodiments, the GABA_{A} receptor negative allosteric modulator or inverse agonist is bemegride, flurothyl, or pentylenetetrazol. In some embodiments, the a5 subunit containing GABA_{A} receptor selective compound is basmisanil, α5IA (3-(5-methylisoxazol-3-yl)-6-[(1-methyl-1H-1,2,3-triazol-4-yl)methoxy][1,2,4]triazolo[3,4-a]phthalazine), L-655,708, MRK-016, PWZ-029, a Pyridazine, Ro4938581, TB-21007, FG-7142 (N-Methyl-9H-pyrido[5,4-b]indole-3-carboxamide), Ro16-0154 (ethyl 7-iodanyl-5-methyl-6-oxo-4H-imidazo[1,5-a][1,4]benzodiazepine-3-carboxylate), Radequinil, Ro15-4513 (Ethyl-8-azido-5,6-dihydro-5-methyl-6-oxo-4H-imidazo-1,4-benzodiazepine-3-carboxylate), Sarmazenil, Suritozole, Terbequinil, or ZK-93426 (ethyl-5-isopropoxy-4-methyl-beta-carboline-3-carboxylate). In some embodiments, the GABA receptor modulator is salvinorin A.

In some embodiments, the methods provided herein are used to treat benzodiazepine dependence, withdrawal, or toxicity, or any combination thereof. In some embodiments, the benzodiazepine dependence, withdrawal, and/or toxicity has resulted from the subject ingesting or being administered adinazolam, alprazolam, bentazepam, bretazenil, bromazepam, bromazolam, brotizolam, camazepam, chlorodiazepoxide, cinazepam, cinolazepam, clobazam, clonazepam, clonazolam, clorazepate, clotiazepam, cloxazolam, delorazepam, diazepam, diclaepam, estazolam, ethyl carfluzepate, ethyl loflazepate, flualprazolam, flubromazepam, flubromazolam, flunitrazepam, flunitrazolam, flutazolam, flutoprazepam, halazepam, ketazolam, loprazolam, lorazepam, lormetazepam, meclonazepam medazepam, mexazolam, midazolam, nifoxipam, nimetazepam, mitemazepam, nitrazepam, nitazolam, mordiazepam, norflurazepam, oxazepam, phenazepam, pinazepam, prazepam, premazepam, pyrazolam, quazepam, rilmazafone, temazepam, tetrazepam, triazolam, or any combination thereof.

In some embodiments, the methods provided herein are used to treat alcohol dependence, withdrawal, or toxicity, sedative dependence, withdrawal, or toxicity, hypnotic dependence, withdrawal, or toxicity, anxiolytic dependence, withdrawal, or toxicity, panic disorder, generalized anxiety disorder, post-traumatic stress disorder (PTSD), idiopathic hypersomnia, narcolepsy, a mood disorder, major depression, dysthymia, chronic suicidality, anxiety disorder NOS, obsessive compulsive disorder, eating disorder NOS, anorexia nervosa, bulimia nervosa, intermittent explosive disorder, a sleep disorder, insomnia NOS, a pain disorder, or a chronic pain disorder.

In some embodiments, the methods provided herein are used to treat alcohol dependence, withdrawal, or toxicity.

In some embodiments, the methods provided herein are used to treat sedative dependence, withdrawal, or toxicity.

In some embodiments, the methods provided herein are used to treat hypnotic dependence, withdrawal, or toxicity.

In some embodiments, the methods provided herein are used to treat anxiolytic dependence, withdrawal, or toxicity.

In some embodiments, the methods provided herein are used to treat panic disorder.

In some embodiments, the methods provided herein are used to treat generalized anxiety disorder.

In some embodiments, the methods provided herein are used to treat post-traumatic stress disorder (PTSD).

In some embodiments, the methods provided herein are used to treat idiopathic hypersomnia.

In some embodiments, the methods provided herein are used to treat narcolepsy.

In some embodiments, the methods provided herein are used to treat mood disorders.

In some embodiments, the methods provided herein are used to treat dysthymia.

In some embodiments, the methods provided herein are used to treat chronic suicidality.

In some embodiments, the methods provided herein are used to treat anxiety disorder NOS.

In some embodiments, the methods provided herein are used to treat obsessive compulsive disorder.

In some embodiments, the methods provided herein are used to treat an eating disorder. In some embodiments, the eating disorder is anorexia nervosa, bulimia nervosa, or eating disorder NOS.

In some embodiments, the methods provided herein are used to treat intermittent explosive disorder.

In some embodiments, the methods provided herein are used to treat insomnia.

In some embodiments, the methods provided herein are used to treat pain disorder.

In some embodiments, the pharmaceutical compound (e.g flumazenil), or an enantiomer, a mixture of enantiomers, or an isotopic variant thereof; or a pharmaceutically acceptable salt, solvate or hydrate thereof, is administered subcutaneously by injection (e.g., bolus or infusion) or by a wearable mini-pump. In some embodiments, the pharmaceutical compound (e.g flumazenil), or an enantiomer, a mixture of enantiomers, or an isotopic variant thereof; or a pharmaceutically acceptable salt, solvate or hydrate thereof, is administered by a wearable mini-pump. In some embodiments, the wearable mini-pump is a 2-day pump. In some embodiments, the wearable mini-pump is a 3-day pump. In some embodiments, a single wearable mini-pump is used.

### EXAMPLES

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that the following claims define the scope of the invention and that methods and structures within the scope of these claims and their equivalents be covered thereby.

### Example 1. Subcutaneous Formulations of Flumazenil

A number of studies from the 1990s indicated have indicated that flumazenil can be useful in the management of withdrawal symptoms following cessation of benzodiazepine use. It has been observed that dosages of ˜1-2 mg of flumazenil administered as a bolus over a period of several hours was able to reduce withdrawal symptoms in high dose benzodiazepine patients who had been abstinent from benzodiazepines for a period of at least three weeks. It has also more recently been determined such bolus administrations in conjunction with benzodiazepine tapering resulting in reduction of benzodiazepine withdrawal symptoms, reduced craving, and increased completion of cessation of benzodiazepines without relapse. Even more recently, it has been observed that continuous dosing at 2 mg/24 h over a continuous period of 96 h by IV with oxazepam tapering successfully managed to drastically curtail withdrawal symptoms over the dosing period.

Thus, currently envisioned treatments of benzodiazepine withdrawal using flumazenil typically would involve in-office use with intravenous delivery being the most likely delivery method. Intravenous delivery can require significant monitoring efforts, IV placement, and various other mechanical requirements of IV procedure (e.g., normal saline, sedatives such as midazolam or propofol, hospital bed or IV chair). Additionally, the flumazenil's poor oral bioavailability and rapid metabolism in the liver (half-life˜4-11 minutes) makes the compound ill-suited to oral dosage forms.

Although IV flumazenil treatment of benzodiazepine dependence and withdrawal can be quite effective, this mode of treatment is only available in the hospital and clinic, creates a substantial amount of medical waste, is costly, requires a meaningful time commitment for patients and healthcare providers alike, and is sometimes limited by compromised venous access. Additionally, the time frame for full benefits of flumazenil treatment to be realized in benzodiazepine withdrawal or dependent patients is not established but is expected to be longer or substantially longer than the approximately five days of infusion offered in the hospital and clinic based detoxification programs. This expectation is based upon a number of reasonable considerations. These include reports of patients in said programs who experienced a benefit from active flumazenil infusions developing uncomfortable withdrawal symptoms upon discontinuation of flumazenil infusions and release to home. It is also reasonable to expect that flumazenil's various potential effects upon the GABA_{A} receptor are a substantial aspect of its beneficial effects in acute benzodiazepine withdrawal, but that for the full advantageous effects of these actions to be fully realized substantial and sustained upregulation post-synaptic receptor GABA_{A} receptor densities may be required.

Based upon the current art and understanding of the processes and times frames involved in substantial and sustained increases in neurophysiologic receptor densities in response to medication, it is reasonable to expect that the full benefit of substantial and sustained upregulation of post-synaptic receptor GABA_{A} receptor densities would not be expected to reach full effect over only five days. Additionally, the comfort of the patient might be reasonably expected to be slowed by the taper of the benzodiazepine or sedative from 5 days to approximately 10 to 90 days in order to decrease the development of acute withdrawal symptoms known to be associated with rapid benzodiazepine taper. Additionally, the comfort of the patient might be reasonably expected to be slowed by the taper of the benzodiazepine or sedative from 5 days to approximately 10 to 90 days in order to decrease the development of post-acute withdrawal symptoms known to be associated with rapid benzodiazepine taper.

Taking all of these factors into account it is reasonable to expect that a sustained 30 to 90 days flumazenil infusion in coordination with a moderate or slow tapering of the benzodiazepine or sedative over a similar time period of around 30 to 90 days would increase patient comfort, or decrease acute withdrawal symptoms, or decrease post-acute withdrawal symptoms, or increase chance of acute treatment success, or decrease rates of relapse. A treatment program or process as described is essentially not possible in the current art due to the costs associated with long hospital stays or at home IV infusions, the substantial dangers of infection associated with long term IV placement, the labs required and dangers associated with long term high volume IV fluid infusions and the notable production of expensive medical waste requiring special disposal. Thus, a subcutaneous formulation that could be administered at home by the patient would offer significant improvements. However, commercially available preparations are rather acidic, having a pH of ˜4.0, which would lead to substantial injection site pain if used as is. Additionally, the limited solubility of flumazenil even under such acidic conditions (˜0.1 mg/mL) further limits the potential utility of a subcutaneous formulation, as the required continuous or near-continuous administration would require infusion of a substantial volume of any such formulation in order to achieve the ˜2-4 mg/day dose required for beneficial effect.

Thus, the formulations provided herein having an elevated concentration of flumazenil (up to ˜10 mg/mL) at a physiologically tolerable pH (˜5-8) and physiologically compatible osmolality (˜300 mOsm/kg) offer substantial benefits and would be amenable to subcutaneous administration. Such formulations allow the patient to self-administer the treatment throughout a prescribed regimen, which can last up to ˜90 days or more in order to treat benzodiazepine withdrawal, while administering from a small device (e.g. a device having a reservoir of mL) that has minimal impact on the lifestyle of the user. Such a formulation would also reduce injection site pain associated with other formulations at lower pH's or elevated osmolalities.

Another advantage provided by the formulations and methods disclosed herein is administration of one or more doses of a drug such as flumazenil according to a programmed dosage regimen. Such devices allow drug administration in the traditional hospital or clinic setting, but also provide the option to self-administer at home or outside the hospital/clinic setting. A doctor or healthcare provider can program a delivery device with a dosage regimen, and the patient or subject is able to use the device to self-administer one or more doses at home. The subject is thus given limited control to implement the pre-programmed dosage regimen. The use of the pre-programmed dosage regimen to self-medicate outside of the clinic allows accurate titration of blood levels with minimally effective doses of an active ingredient such as flumazenil. This can decrease the procedural burden and medical equipment required during treatment in the clinic or hospital preformed through the current art consisting of IV infusion or IM bolus injection. Moreover, the dosage regimen can be programmed to control the rate of drug delivery to mitigate certain side effects known to be associated with flumazenil usage. In some cases, the dosage regimen is programmed for sustained release and/or extended release dosing of a drug such as flumazenil.

Additionally, the formulations and method provided herein allow a medical professional to implement a pre-programmed dose regimen of flumazenil that can be tailored the specific needs of a patient, particularly depending on the manner in which the patient is ceasing use of benzodiazepine or sedative use. For example, a patient undergoing a taper of benzodiazepine may require only a minimal dosage of flumazenil for the first stage of the taper where the dose of benzodiazepine will still be high. In such a case, during this early stage of the taper, less flumazenil or other GABA receptor modulator compound may be required as withdrawal symptoms are likely to be less severe at this stage. The daily dosage of flumazenil can thus be tailored to the taper of the benzodiazepine such that only as much flumazenil as is necessary is administered at a given time, with more flumazenil being administered as the taper continues and the patient receives a lower dose of benzodiazepine. Additionally, the dose of flumazenil itself can be tapered off near the regimen in order to avoid or minimize any effects of potential flumazenil withdrawal. Any such pre-programmed schedule can be accommodated as required by the prescribing medical professional.

### Example 2. Testing the Formulation Disclosed Herein

1) The formulations provided herein, in comparison to standard flumazenil, are tested to determine an association with reduced subjective experience of erythema and/or pain and/or itching and/or burning and/or stinging and/or irritation and/or other measured parameters signifying a noxious effect to the administered tissues in acute and/or chronic testing in animals and/or humans will be demonstrated. Animal data could be obtained by treating one or two cohorts of animals, one treated subcutaneously by our formulation of flumazenil, and another treated subcutaneously by currently available flumazenil USP. Animal technicians would observe and document signs of irritation and/or erythema in the local site dermal tissue. Local site irritation and erythema are accurate markers of tissue damage and generally also associated with increased pain.

2) The formulations provided herein, in comparison to standard flumazenil USP, are tested to determine an association with reduced histological changes and/or signs of tissue damage and/or denigration and/or desiccation and/or inflammation in in-vitro and/or in-vivo human and/or animal tissues will be demonstrated. Histology data could be obtained by observing dermal tissue samples collected from one or two cohorts of animals, one treated subcutaneously by our formulation of flumazenil, and another treated subcutaneously by currently available flumazenil USP. Biopsy specimens from each cohort would undergo fixation, processing and staining before observation by a trained histotechnologist.

3) The formulations provided herein are tested to determine that the formulation successfully holds in solution and/or complexes and/or solubilizes and/or emulsifies any or all of the flumazenil that is present at the higher pH than standard flumazenil USP formulations will be demonstrated. Flumazenil solubility curves in the presence of a complexing agent possibly to include any of the described agents in this document and/or potentially to include a sulfobutylether-β-cydodextrin (e.g., Captisol®) or a hydroxypropyl-β-cydodextrin (e.g., Cavasol®) is predicted to establish the percentage of flumazenil that is successfully held in solution at a given target pH, the achievement of which would be impossible without said complexing agent.

4) The formulations of flumazenil provided herein are tested to determine that the concentration of flumazenil that can be maintained in solution.

5) The formulations of flumazenil provided herein are tested to demonstrate performance with fidelity across delivery parameters identified in the pump chosen for formulation delivery in comparison to the performance of this same pump delivering medication for which it was initially FDA approved (potentially to include standard insulin or another medication or a compound or a “biologic” medication, or a molecule or a solution).

6) The formulations provided herein, in comparison to standard of care, are tested to determine their association with comparably reduced subjective and/or objective signs or symptoms of benzodiazepine withdrawal or dependence.

7) The formulations provided herein, in comparison to placebo, are tested to determine their association with increased reductions in subjective and/or objective signs or symptoms of benzodiazepine withdrawal or dependence, including identified clinical symptoms in comparison to placebo.

8) The formulations provided herein, in comparison to standard flumazenil, are tested to determine that they are more stable and effective at maintaining all flumazenil in solution at a higher pH than currently available flumazenil at a higher concentration.

9) The formulations provided herein are tested to determine if they successfully complex and/or solubilize and/or emulsify any flumazenil than that of standard USP flumazenil formulations. Standard flumazenil is formulated to be at an acidic pH (˜4) for maximum solubility without any excipient emulsifying agents, complexing agents, surfactant agents or solubilizing agents. Thus, it is predicted that data will be provided demonstrating that a stable flumazenil solution formulation has been developed as proposed that achieves an average pH that is at least 1 pH point higher (and may be at least 2-3 pH points higher) than the average found in current standardized USP formulations of flumazenil of comparable strength or even lower strength.

10) Solubilizing agents will be tested to characterize flumazenil solubility curves within different concentrations of solubilizing agents potentially including but not limited to sulfobutyl-ether-beta-cyclodextrin (e.g., Captisol®), hydroxypropyl-beta-cyclodextrin or other cyclodextrin entities will be provided

### Example 3. Potential Initial Formulation Protocol for Flumazenil

Dissolve flumazenil, HP-beta-CD and potential buffer (TBD) in sterile water. Dilute with water to 90% of final target volume. Adjust the pH to the target range with aqueous NaOH prepared in sterile water. Adjust to the final volume by the addition of sterile water.

### 1) Initial Testing:

a. Solubility: Determine the solubility of flumazenil at different SBE-β-CD and HPBCD concentrations: The solubility of SBE-β-CD is reported as 0.6 g/mL and HPBCD is similarly very soluble. Between 10 to 600 mg/mL of either cyclodextrin should complex the flumazenil in solution at a potential total flumazenil concentration range of 5 to 10 mg/ml. This solubility should be independent of pH, and thus the system can be buffered to a physiologically compatible pH of ˜7-8 The actual pH of the final solution and concentrations of SBE-β-CD/HPBCD, flumazenil, and any optional preservative and/or added buffer(s) necessary to achieve formulation goals is to be determined.
b. Stability Testing: Solutions made in this initial phase should be stored at controlled room temperate for one month and three months to determine initial stability by Appearance, pH, osmolality and flumazenil and SBE-β-CD (e.g., Captisol®) and/or HPBCD concentrations.
   i. Short Term
      1. Validation of concentration with HPLC or other means
      2. pH
      3. Osmolality
      4. Appearance (precipitation of flumazenil)
      5. Also determine ICH compliant stress testing results (light, heat, pH, etc.)
   ii. Long Term (≥6 months)
      1. Stability in Temperature Cycling (freeze/thaw cycles)
      2. Anti-Microbial Properties/Susceptibility to Contamination.
      3. Short and Longer-term Stability in Cyclic Olefin Polymer capsules, device cartridges and USP glass

### 3) Buffers

a. It is believed that flumazenil has a solubility curve that is not substantially dependent on pH, and thus addition of a buffer may be added for pH stability,
b. Only non-irritating buffers will likely be considered, including phosphates and histidine.

### 4) Emulsifiers

a. SBE-β-CD (e.g., Captisol®)—This is a top choice given that it is an industry standard, substantial safety data already exists, and the parent company can provide support. Drawbacks include high osmolality at elevated concentration due to presence of sodium ions. However, this drawback can be overcome by preparing a free acid derivative, which can then form counterions with either a flumazenil itself orb) appropriate buffer added to the formulation.
b. Hydroxypropyl-beta-cydodextrin/HP-b eta-CD (HPBCD)—This is also a high choice at it is commonly used in the industry and there is substantial safety data available. However, the safety profile of HPBCD is slightly inferior to SBE-β-CD which could raise complications.
c. Other possibilities:
   i. Polysorbate/Tween 20 or Tween 80
   ii. Glycerin
   iii. Propylene glycol
   iv. Complexing/emulsifying agent
      5) Current Proposed Label (Subject to Confirmation after Testing and Finalization)
Flumazenil for Subcutaneous Injection
Rx Only
pH 5.0-8.0
Strength: potential range 3-10 mg/mL
Ingredients:
   Flumazenil potential range 3-10 mg/mL
   Cyclodextrin (SBE-β-CD or HPBCD) potential range 10 mg/mL to 300 mg/mL
   Benzethonium chloride potential range 0.1-0.5 mg/mL
   Buffer (TBD)
   Store at 20° to 25° C. (Controlled Room Temperature) or at 2° to 8° C.
      (Refrigerated) dependent on further testing

### Example 4. Potential Dosing Regimen for Flumazenil Formulation

A patient is either diagnosed with benzodiazepine dependence or suffering from benzodiazepine withdrawal. The patient either has stopped administering or ingesting benzodiazepines or is undergoing a tapering down of benzodiazepine regimen.

The patient is prescribed a regimen of flumazenil by subcutaneous injection. The regimen is to include 3 mg/day of flumazenil at continuous dosing fora period of 30 days, during which the subject will receive a tapering of benzodiazepine or will abstain from taking any benzodiazepine.

The prescription includes 10 wearable pump devices configured to deliver the flumazenil formulation provided herein at a continuous rate such that the subject receives the prescribed 3 mg/day of flumazenil. Each wearable pump device contains a 3 mL reservoir of the flumazenil formulation provided herein at a concentration of 3.5 mg/mL flumazenil. The patient applies the wearable pump device to their body and initiates the pump to begin the regimen. The pump operates continuously to administer the required volume to deliver the prescribed 3 mg/day flumazenil. On the third day, before the wearable pump device has ejected the full volume of the reservoir, the patient removes the first device and applies a second device, then resumes the regimen. The is repeated for each of the 10 devices until the dosing regimen is completed.

### Example 5. Preparation of Flumazenil Salt Solution with Captisol Acid

A number of methods were tested to prepare a flumazenil salt solution as shown below.

To prepare a Captisol Acid-Flumazenil salt solution, Captisol acid (6.26% moisture, 8.14 mg active, 8.70 mg M.C.) was dissolved in HPLC grade H₂O in a test tube and stirred vigorously. A total of 8.0 mg of flumazenil was weighed out and added in portions to the Captisol acid solution. The first portion (˜2 mg) was added and stirred vigorously. The flumazenil did not immediately dissolve, but went into solution after ˜30 min. A second portion (˜2 mg) was then added and stirred, but substantial solids remained insoluble. The mixture was stirred overnight and solid was still suspended in solution the next day.

To prepare a Captisol-Flumazenil Complex via organic solvent, a 20 mL glass scintillation vial was charged with a magnetic Teflon stirbar and a solution of flumazenil (10 mg) dissolved in MeOH (10 mL) was added. Captisol (5.53% moisture, 214 mg active, 227 mg M.C.) was added and the suspension was stirred at rt until all solids went into solution (˜30 min). The solution was added to a beaker and allowed to evaporate overnight resulting in a white, fluffy solid (214 mg). A portion of solid (90 mg) did not dissolve into HPLC grade H₂O (0.5 mL). Another portion of HPLC grade H₂O (0.5 mL) was added, but the solid still did not visibly dissolve. Heating of the solution from the bottom with a heat gun caused the solid to dissolve, but a solid crystalized upon cooling back to room temperature. These crystals had the same appearance as flumazenil crystallized from H₂O.

To prepare Flumazenil-γ-Cyclodextrin complex, a solution of γ-cydodextrin (200 mg) was dissolved in HPLC grade H₂O to make 1 mL of a 20% solution in a volumetric flask. Flumazenil (1.1 mg) was then added and stirred. While initially the flumazenil dissolved to give a transparent solution, the solution became cloudy over time. Sonication and vigorous stirring did not cause the precipitate to dissolve. This prompted the investigation of saturating γ-cyclodextrin solution with flumazenil. An additional portion of flumazenil (49 mg, 50.1 mg total) was added to the cloudy solution and the mixture was stirred with a magnetic Teflon stirbar for 48 h at room temperature. The suspension was filtered through a 0.45 μm syringe filter and diluted in duplicate to determine the concentration of flumazenil by HPLC. [Flumazenil]: 2.41 mg/mL.

To prepare another flumazenil solution, hydroxypropyl-β-cydodextrin (800 mg) was dissolved in HPLC grade H₂O in a volumetric flask to make 2 mL of a 40% solution. Flumazenil (50 mg) and a magnetic Teflon stirbar were added and the test tube sealed with parafilm, covered in aluminum foil, and allowed to stir for 48 h at room temperature. The mixture was then filtered through a 0.45 μm syringe filter and osmolality measured. The sample was also diluted in duplicate to determine the concentration of flumazenil by HPLC. Osmolality: 652 mOsm/kg [Flumazenil]: 3.8 mg/mL.

To prepare a Captisol-Ethylenediamine-Flumazenil complex solution without pH adjustment, Captisol-ethylenediamine (40% Captisol acid equivalent) was prepared by titrating a solution of Captisol acid in HPLC grade H₂O with ethylenediamine (0.5 molar equivalent). Specifically, Captisol acid (6.41% moisture, 2.0 g active, 2.14 g M.C.) was dissolved in HPLC grade H₂O (3.5 mL) and ethylene diamine (215 μL, 193 mg) was added in 5 portions via micropipette. The solution was allowed to stir for ˜2 min between aliquot additions. When the addition was complete, the solution was diluted with HPLC grade H₂O to a final volume of 5 mL in a volumetric flask. The solution was diluted to 30% Captisol (acid equivalents) in a test tube and flumazenil (7.5 mg) was added to make a target concentration of 1.5 mg/mL. The transparent, yellow solution was sealed with parafilm, covered in aluminum foil, and stirred for 48 h at rt. The solution was filtered through a 0.45 μm syringe filter and osmolality measured. The sample was also diluted in duplicate to determine the concentration of flumazenil by HPLC and this was repeated three times. The sample was stored in a parafilm sealed 20 mL glass scintillation vial and covered in aluminum foil to protect from light. Final pH: 6.41. Osmolality: 702 mOsm/kg [Flumazenil]: 1.15±0.05 mg/mL.

To prepare a Captisol-Ethylenediamine-Flumazenil Complex with pH adjustment, Captisol acid (8.81% moisture, 1.50 g active, 1.64 gM.C.) was dissolved in HPLC grade H₂O (3 mL) in a test tube with a magnetic Teflon stirbar and stirred vigorously via magnetic stirring. Ethylenediamine (161 μL, 144.9 mg) was added in a single portion with stirring followed by adding benzethonium chloride (50 μL of 1% solution, final concentration 0.01%). The solution was then titrated with 2M HCl (90 μL total) until a final pH of 4.99. The solution was diluted to a final volume of 5 mL in a volumetric flask and then flumazenil (7.6 mg) was added directly to the volumetric flask. The mixture was sealed with parafilm, covered in aluminum foil, and allowed to stand at room temperature for 48 h with periodic agitation. The mixture was filtered through a 0.45 μm syringe filter and osmolality was measured. The sample was also diluted in duplicate to determine the concentration of flumazenil by HPLC and repeated three times. The solution was stored in a parafilm sealed scintillation vial and covered in aluminum foil to protect from light. Osmolality: 875.3 mOsm/kg. [Flumazenil]: 1.35±0.07 mg/mL. Final pH: 4.99.

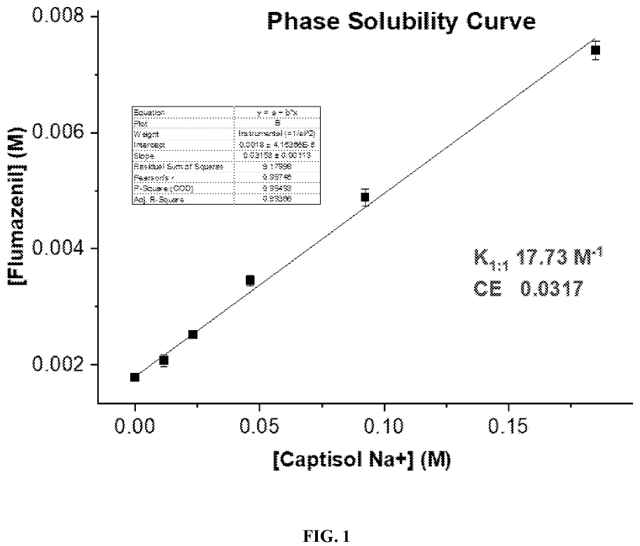

The phase solubility curve with Captisol and Flumazenil was shown in FIG. 1. The different formulations of flumazenil were listed in Table 1.

TABLE 1

| | [Flumazenil] | Osmolality | |
|---|---|---|---|
| Formulation | (mg/mL) | (mOsm/kg) | pH |
| 30% Captisol- | 1.15 | 702 | 6.41 |
| Ethylenediame | | | |
| (CapAcid equiv) | | | |
| pH 6.41 | | | |
| 30% Captisol- | 1.35 | 873.3 | 4.99 |
| Ethylenediame | 1.39 (1 Month | | |
| (CapAcid equiv) | Stability) | | |
| pH 4.99 | | | |
| 40% Hydroxypropyl-β- | 3.80 | 652 | ND |
| cyclodextrin | | | |
| 20% γ-cyclodextrin | 2.41 | ND | ND |
| 40% Captisol | 2.24 | ND | ND |
| (Phase solubility) | | | |
| 20% Captisol | 1.48 | ND | ND |
| (Phase solubility) | | | |

ND: Not Determined

Although the disclosure has been described with reference to the above examples, it will be understood that modifications and variations are encompassed within the spirit and scope of the disclosure. Accordingly, the disclosure is limited only by the following claims.

What is claimed is:

1. A pharmaceutical composition, comprising:
(i) flumazenil, or an isotopic variant thereof; or a pharmaceutically acceptable salt, solvate or hydrate thereof; and
(ii) at least one pharmaceutically acceptable excipient,
wherein the pharmaceutical composition is in a form for dosing or administration by subcutaneous injection; and
wherein the concentration of flumazenil in the pharmaceutical composition is greater than 0.7 mg/mL.

2. The pharmaceutical composition of claim 1, wherein the at least one pharmaceutically acceptable excipient comprises a complexing agent.

3. The pharmaceutical composition of claim 2, wherein the complexing agent is a substituted or un substituted cyclodextrin.

4. The pharmaceutical composition of claim 3, wherein the complexing agent is a cyclodextrin substituted with at least one acidic functional group.

5. The pharmaceutical composition of claim 4, wherein the complexing agent is a sulfobutyl-ether-beta-cyclodextrin (SBEBCD).

6. The pharmaceutical composition of claim 1, wherein the molar ratio of complexing agent to flumazenil is from about 1:10 to about 10:1.

7. The pharmaceutical composition of claim 1, wherein the pharmaceutical composition further comprises an emulsifying agent, a surfactant, a solubilizing agent, a co-solvent or a combination thereof.

8. The pharmaceutical composition of claim 7, wherein the co-solvent is ethanol, propylene glycol, tween 20, tween 80, or glycerin.

9. The pharmaceutical composition of claim 1, wherein the pharmaceutical composition has a pH of about 5 to about 8.

10. The pharmaceutical composition of claim 1, wherein the concentration of flumazenil in the pharmaceutical composition is at least about 1 mg/mL.

11. The pharmaceutical composition of claim 1, wherein the pharmaceutical composition has an osmolality of from about 300 mOsm/kg to about 500 mOsm/kg.

12. The pharmaceutical composition of claim 1, wherein the pharmaceutical composition is adapted to be delivered to a subject over a period of at least two days.

13. The pharmaceutical composition of claim 12, wherein the pharmaceutical composition is adapted to deliver a daily dose of at least 1 mg of flumazenil.

14. The pharmaceutical composition of claim 12, wherein the pharmaceutical composition is adapted to be delivered by a wearable device.

15. The pharmaceutical composition of claim 14, wherein the wearable device has a reservoir of the pharmaceutical composition of less than 10 mL.

16. A method of treating benzodiazepine dependence, withdrawal, or toxicity in a subject in need thereof, the method comprising administering a therapeutically effective amount of the pharmaceutical composition of any one of claims 1-15.

17. The method of claim 16, wherein the benzodiazepine withdrawal is acute withdrawal or post-acute withdrawal.

18. A method of treating alcohol dependence, withdrawal, or toxicity, sedative dependence, withdrawal, or toxicity, hypnotic, withdrawal, dependence or toxicity, anxiolytic dependence, withdrawal, or toxicity, panic disorder, generalized anxiety disorder, post-traumatic stress disorder (PTSD), idiopathic hypersomnia, narcolepsy, a mood disorders, major depression, dysthymia, chronic suicidality, anxiety disorder NOS, obsessive compulsive disorder, eating disorder NOS, anorexia nervosa, bulimia nervosa, intermittent explosive disorder, a sleep disorder, insomnia NOS, a pain disorder, or a chronic pain disorder in a subject in need thereof, the method comprising administering a therapeutically effective amount of the pharmaceutical composition of any one of claims 1-15.

19. A method of treating a disease or disorder in a subject in need thereof, comprising administering to the subject a therapeutically effective amount of flumazenil, wherein the flumazenil is administered by subcutaneous injection in an amount of at least 0.5 mg/day from a single device for a period of time of at least 2 days, wherein the single device has a reservoir volume less than 30 mL.

20. The method of claim 19, wherein the disease or disorder is benzodiazepine dependence, withdrawal, or toxicity, alcohol dependence, withdrawal, or toxicity, sedative dependence, withdrawal, or toxicity, hypnotic dependence, withdrawal, or toxicity, anxiolytic dependence, withdrawal, or toxicity, panic disorder, generalized anxiety disorder, post-traumatic stress disorder (PTSD), idiopathic hypersomnia, narcolepsy, a mood disorder, major depression, dysthymia, chronic suicidality, anxiety disorder NOS, obsessive compulsive disorder, eating disorder NOS, anorexia nervosa, bulimia nervosa, intermittent explosive disorder, a sleep disorder, insomnia NOS, a pain disorders, or a chronic pain disorder.

21. The method of claim 19, wherein the disease or disorder is benzodiazepine dependence, withdrawal, or toxicity.

22. The method of claim 21, wherein the benzodiazepine withdrawal is acute withdrawal or post-acute withdrawal.

23. The method of any one of claims 19-22, wherein the flumazenil is administered as a pharmaceutical composition having a concentration of flumazenil of greater than 0.7 mg/mL.

24. The method of any one of claims 19-23, wherein the flumazenil is administered in an amount of at least 0.5 mg/day, at least 1.0 mg/day, at least 1.5 mg/day, at least 2 mg/day, at least 3 mg/day, at least 4 mg/day, at least 5 mg/day, at least 7.5 mg/day, or at least 10 mg/day.

25. The method of any one of claims 19-24, further comprising administering to the subject a therapeutically effective amount of flumazenil from one or more additional single devices, wherein each additional single device is administered to the subject after another iteration of the period of time.

26. The method of any one of claims 18-25, wherein the flumazenil is administered continuously.

27. The method of claim 28, wherein the flumazenil is administered at a rate of at least about μg/hr, at least about 60 μg/hr, at least about 80 μg/hr, at least about 100 μg/hr, at least about 150 μg/hr, at least about 200 μg/hr, or at least about 250 μg/hr.

28. A pharmaceutical composition, comprising:
(i) a pharmaceutical compound, wherein the pharmaceutical compound is a GABA_{A} receptor antagonist or modulator, a GABA receptor agonist, a GABA receptor partial agonist, a GABA_{A} receptor negative allosteric modulator or inverse agonist, or salvinorin A, or an isotopic variant thereof; or a pharmaceutically acceptable salt, solvate or hydrate thereof; and
(ii) at least one pharmaceutically acceptable excipient, wherein the at least one pharmaceutically acceptable excipient comprises a cyclodextrin substituted with at least one acidic functional group or a conjugate base thereof or a cyclodextrin substituted with at least one polar functional group,
wherein the pharmaceutical composition is in a form for dosing or administration by subcutaneous injection.

29. The pharmaceutical composition of claim 28, wherein the pharmaceutical compound is a GABA_{A} receptor antagonist or modulator.

30. The pharmaceutical composition of claim 29, wherein the pharmaceutical compound is flumazenil or pentylenetetrazol.

31. The pharmaceutical composition of claim 28, wherein the pharmaceutical compound is a GABA receptor agonist.

32. The pharmaceutical composition of claim 31, wherein the pharmaceutical compound is muscimol, thiomuscimol, or gaboxadol.

33. The pharmaceutical composition of claim 28, wherein the pharmaceutical compound is a GABA receptor partial agonist.

34. The pharmaceutical composition of claim 33, wherein the pharmaceutical compound is bretazenil, imidazenil, FG 8205 (7-chloro-5-methyl-3-(5-propan-2-yl-1,2,4-oxadiazol-3-yl)-4H-imidazo[1,4]benzodiazepin-6-one), abecarnil, NS 2710 (1-[1-[3-(3-pyridyl)phenyl]benzimidazol-5-yl]ethanone O-ethyloxime), RWJ-51204 (5-ethoxymethyl-7-fluoro-3-oxo-1,2,3,5-tetrahydrobenzo[4,5] imidazo[1,2a]pyridine-4-N-(2-fluorophenyl)carboxamide), or premazepam.

35. The pharmaceutical composition of claim 28, wherein the pharmaceutical compound is a GABA_{A} receptor negative allosteric modulator or inverse agonist.

36. The pharmaceutical composition of claim 35, wherein the pharmaceutical compound is bemegride, flurothyl, or pentylenetetrazol.

37. The pharmaceutical composition of claim 28, wherein the pharmaceutical compound is an a5 subunit containing GABA_{A} receptor selective compound.

38. The pharmaceutical composition of claim 37, wherein the pharmaceutical compound is basmisanil, α5IA (3-(5-methylisoxazol-3-yl)-6-[(1-methyl-1H-1,2,3-triazol-4-yl)methoxy] [1,2,4]triazolo[3,4-a]phthalazine), L-655,708, MRK-016, PWZ-029, a Pyridazine, Ro4938581, TB-21007, FG-7142 (N-Methyl-9H-pyrido[5,4-b]indole-3-carboxamide), Ro16-0154 (ethyl 7-iodanyl-5-methyl-6-oxo-4H-imidazo[1,5-a][1,4]benzodiazepine-3-carboxylate), Radequinil, Ro15-4513 (Ethyl-8-azido-5,6-dihydro-5-methyl-6-oxo-4H-imidazo-1,4-benzodiazepine-3-carboxylate), Sarmazenil, Suritozole, Terbequinil, or ZK-93426 (ethyl-5-isopropoxy-4-methyl-beta-carboline-3-carboxylate).

39. The pharmaceutical composition of claim 28, wherein the pharmaceutical compound is salvinorin A.

40. A method of treating a disease or disorder in a subject in need thereof, comprising administering to a subject in need thereof a therapeutically effective amount of a pharmaceutical compound, wherein the pharmaceutical compound is GABA_{A} receptor antagonist or modulator, a GABA receptor agonist, a GABA receptor partial agonist, a GABA_{A} receptor negative allosteric modulator or inverse agonist, or salvinorin A, wherein the pharmaceutical compound is administered by subcutaneous injection from a single device for a period of time of at least 2 days, wherein the single device has a reservoir volume less than 30 mL.
